(19) United States
(12) Patent Application Publication
(10) Pub. No.: US 2021/0301247 A1
(43) Pub. Date: Sep. 30, 2021

(54) SYSTEM FOR IMAGE-DRIVEN CELL MANUFACTURING

(71) Applicant: CELLINO BIOTECH, INC., Cambridge, MA (US)

(72) Inventors: Marinna Madrid, Cambridge, MA (US); Matthias Wagner, Cambridge, MA (US); Suvi Aivio, Arlington, MA (US); Stan Wang, Cambridge, MA (US); Edroaldo Lummertz Da Rocha, Cambridge, MA (US)

(21) Appl. No.: 17/267,383

(22) PCT Filed: Aug. 9, 2019
(86) PCT No.: PCT/US2019/045969

§ 371 Date: Feb. 9, 2021

Related U.S. Application Data
(60) Provisional application No. 62/756,141, filed on Nov. 6, 2018.
(60) Provisional application No. 62/739,027, filed on Sep. 28, 2018.
(60) Provisional application No. 62/717,581, filed on Aug. 10, 2018.

(51) Int. Cl. C12M 1/34; G06K 9/00; C12M 1/00; C12N 5/0793; C12N 15/11; C12N 9/22; C12N 15/90; G01N 15/14

(52) U.S. Cl. CPC C12M 41/36; G01N 2015/1006; C12M 23/20; C12N 5/0619; C12M 47/06; C12M 41/38; C12N 15/11; C12N 9/22; C12N 15/907; G01N 15/1468; G01N 15/1434; C12N 2310/20; C12N 2800/80; C12N 2510/00; C12N 2506/45; G06K 9/00147

(57) ABSTRACT

Systems and methods for image-driven cell manufacturing are provided. Systems comprise a substrate for cells, imaging system for imaging cells on the substrate, computing system that computes cell characteristics from images, and a pulsed laser scanning system. The substrate is suitable for high-resolution cell imaging and is coated with a layer that partially absorbs laser pulses for the purpose of converting energy into microbubble formation. The computing system communicatively coupled to the pulsed laser scanning system and directs laser pulses to the substrate under targeted cells. Laser pulses are converted into mechanical energy via microbubbles that, depending on laser energy and pulse pattern, destroy selected cells, remove selected cells, or temporarily porate selected cells for the purpose of introducing biological cargos.

### CROSS-REFERENCE TO RELATED APPLICATIONS

This application claims the benefit of, and priority to, U.S. Provisional Application No. 62/717,581, filed Aug. 10, 2018, U.S. Provisional Application No. 62/739,027, filed Sep. 28, 2018, and U.S. Provisional Application No. 62/756,141, filed Nov. 6, 2018, the contents of each of which are incorporated by reference.

### TECHNICAL FIELD

The invention generally relates to cells, and particularly relates to systems for generation of cell differentiation programs, image-based laser-scanning for applications involving cells, and cell fate specification and targeted maturation.

### BACKGROUND

Stem cells are cells that are characterized by the ability to multiply indefinitely and the ability to develop into many different cell types. Some stem cells even have the potential to develop into any specific cell type. Stem cells are potentially useful in medicine as a source of cells to supplement or replace cells lost to disease. They also have the potential to be differentiated into specific cell types to be used in the production of certain therapeutics of biological origin, such as insulin or clotting factors.

Efforts to differentiate stem cells to specific cell types for use in research or medicine are met with limited success due to the complexity and unpredictability of the biological processes involved. For a few certain cell types, it is understood that certain combinations of growth or transcription factors may be delivered under controlled culture conditions to allow the stem cells to differentiate. However, only a few differentiation pathways have been studied. Due to the unpredictability involved, those few pathways that have been studied do not provide general guidance for differentiating stem cells into various desired specific cell types.

Additionally, the present technology in the field does not allow for their guided maturation to terminally differentiated or fully functional cell types with a minimal set of targeted genes and associated effectors for modulating their expression. The present approach is comprised of manual trial by error, which is time consuming and inefficient. Therefore, there is a need in the art for a more generalized and automated approach to utilizing stem cells to produce specified mature cells, to aid in guiding experiments in biological research and further developments in regenerative medicine.

### SUMMARY

The invention provides high throughput methods and devices for the manufacture of cells that have specific functional and/or morphologic features. The invention is especially useful when thousands, if not hundreds of thousands or millions, of cells are required. At the same time, methods of the invention provide tailored delivery of specific cells, depending on local cell culture conditions including cell density, cell morphology, cell phenotype, cell expression, or a combination thereof. The invention provides the ability to use automatable, physical techniques to manage cellular differentiation and proliferation, which is a key advantage when cells transition through multiple states. In turn, systems of the invention advantageously provide spatial control over delivery parameters in order to enhance efficiency. In some embodiments, systems of the invention allow for the manufacture of tailored, engineered populations of different cell phenotypes.

Laser-activated constructs are used to transfer energy to cells for the purposes of intracellular delivery of cargoes, extraction of cargoes from within cells, and cell killing and/or detachment. Optimal laser irradiation parameters are determined based on cell information, such as cell density, cell type, cell morphology, intracellular protein expression, and surface protein expression. Examples of parameters include, but are not limited to, illuminated area, laser fluence, number of laser pulses, timing between laser pulses, speed of laser scan, number of laser scans, and timing between laser scans.

Methods and workflows for image-based laser-scanning of laser-activated constructs for various applications involving cells are described. The use of laser-activated constructs, light absorbers capable of absorbing laser radiation and transferring the energy to the environment, for intracellular delivery, extraction of cargoes from within cells, and cell killing, cell detachment, or a combination thereof are described. The invention uses various methods for intracellular delivery of large molecules and particles. Using information acquired from images enables the laser-scanning parameters for delivery, extraction, killing, cell detachment, or a combination thereof to be optimized depending on the state of the cells and the intended application.

Substrates used in the invention are suitable for high-resolution cell imaging and converts optical energy into mechanical energy efficiently. Conventional technology requires several orders of magnitude more optical energy, which results in low throughput and potential thermal damage to cell cultures, or requires additives to the cell media to enhance absorption, which is highly undesirable, especially for clinical applications. Systems ad methods of the invention provide high throughput, while avoiding undesirable additives.

In an embodiment, systems of the invention comprise a substrate, an imaging system, a computing system, and a pulsed laser scanning system. The substrate for cells is suitable for high-resolution cell imaging. In some embodiments, the substrate is coated with a layer that partially absorbs laser pulses for the purpose of converting energy into microbubble formation.

The imaging system is provided to image cells on the substrate. The computing system is provided to compute cell characteristics from one or more images. The computing system is used to direct a pulsed laser scanning system. The pulsed laser scanning system directs laser pulses to the substrate under targeted cells, where laser pulses are converted into mechanical energy via microbubbles. The microbubbles, depending on laser energy and pulse pattern, are used to destroy selected cells, remove selected cells, temporarily porate selected cells for the purpose of introducing biological cargos, or a combination thereof.

Embodiments of the invention use laser-based cell poration techniques to extract cargoes and/or proteins of interest from within cells, to kill cells, to detach adherent cells from a surface, or a combination thereof. Methods of the invention deliver membrane-impermeable cargoes into cells while maintaining cell viability. Methods of the invention deliver to entire populations of cells, to subpopulations of cells within a larger sample, and to specific regions within a single cell (i.e. sample, cell-level, and sub-cell resolution). Methods of the invention extract cargoes from within cells while maintaining cell viability. Methods of the invention extract cargoes from entire populations of cells, from subpopulations of cells within a larger sample, and from specific regions within a single cell (i.e. sample, cell-level, and sub-cell resolution). Methods of the invention kill specific cells within a population (i.e. sample and cell-level resolution). Methods of the invention deliver detach specific cells within a population from a surface and also detach specific regions of a specific cell (i.e. sample, cell-level, and sub-cell resolution).

Moreover, embodiments of the invention combine laser-based cell poration with image analysis to optimize laser scanning protocols for intracellular delivery, extraction of cells, killing of cells, and cell detachment. The invention allows for the ability to spatially-selectively porate cells with sub-cell resolution, taking internal cellular organization into consideration (i.e. in application where it is necessary to deliver a cargo to the nucleus, it may be advantageous to porate the cell in the region containing the nucleus). The invention allows for the ability to spatially-selectively porate cells within a population, with cell-level resolution. The invention allows for the ability to modify the amount of energy imparted to the membrane of each cell in a population based on information including, but not limited to: cell density, cell morphology, intracellular and surface protein expression, etc. The invention allows for continuous monitoring of cell state (including cell density, cell morphology, protein expression, etc.) in real time, both to optimize and characterize the effects of poration for intracellular delivery or cargo extraction. The invention allows for compatibility with semi-automated and fully-automated workflows.

In some embodiments, systems and methods of the invention are used to deliver barcodes to cells based on the imaging characteristics of the cells. The delivered barcodes allow those cells to be distinguished from other cells in downstream analysis. For example, barcoding may be useful during single-cell RNA sequencing.

In some embodiments, systems and methods of the invention are used to co-deliver fluorescent indicator molecules together with active biological compounds. Delivery of the biological compounds may then be verified by imaging the fluorescent indicator molecules.

The system may further comprise an absorbing layer. The absorbing layer absorbs transmission at one or more imaging wavelengths of preferably greater than about 25%, about 50%, or about 75%. Wavelengths absorbed are in the range of about 400 nm to about 700 nm. In some embodiments, absorption is at a pulsed laser wavelength of greater than about 5%, about 10%, about 15%, about 25%, or about 50%. In an embodiment, the design features over a majority of the absorbing layer surface are not larger than about 0.25 wavelength, about 0.5 wavelength, about 1.0 wavelength, or about 2.0 wavelength of the imaging light.

The absorbing layer is compatible with cell culture and common cell culture matrices. The absorbing layer does not leach or ablate into cell culture. For example, the absorbing layer may comprise titanium, gold, or a combination thereof. In one embodiment, the absorbing layer is titanium, for example 20 nm. In one embodiment, the absorbing layer is gold, for example 10 nm. In one embodiment, the absorbing layer is titanium and gold, for example at 5 nm and 5 nm. In one embodiment, the absorbing layer is 50 nm gold nanoparticles dispersed and attached to the substrate.

The system may further comprise an antireflection layer on a side of the substrate opposite the surface on which cells are disclosed. In an embodiment, the antireflection layer has reflection of less than 2% at pulsed laser wavelength. In an embodiment, the antireflection layer has reflection of less than 5% at imaging wavelength(s). For example, the antireflection layer may have a lambda/4 MgF2 broadband coating. In an embodiment, the substrate may go through transmission at imaging wavelengths of greater than about 75%, greater than about 90%, or greater than about 95%.

In an embodiment, the laser may comprise a pulse width of less than or equal to about 100 nsec, less than or equal to about 50 nsec, less than or equal to about 25 nsec, less than or equal to about 15 nsec, less than or equal to about 10 nsec, less than or equal to about 5 nsec, or less than or equal to about 1 nsec. In an embodiment, the laser pulse frequency comprises greater than or equal to about 10 kHz, greater than or equal to about 20 kHz, greater than or equal to about 100 kHz, greater than or equal to about 150 kHz, greater than or equal to about 500 kHz, or greater than or equal to about 1 MHz. In an embodiment, the invention comprises a pulse fluence at a substrate of less than or equal to about 500 mJ/cm², less than or equal to about 250 mJ/cm², less than or equal to about 100 mJ/cm², less than or equal to about 75 mJ/cm², or less than or equal to about 50 mJ/cm² during cell processing. In an embodiment, the invention comprises a laser spot diameter at a substrate of less than or equal to about 100 μm, less than or equal to about 50 μm, less than or equal to about 20 μm, less than or equal to about 10 μm, or less than or equal to about 5 μm.

The invention provides systems and methods for directing cell fate and allows the use of minimal target/effector combinations to direct differentiation of stem cells. Genomic targets that promote differentiation of a desired cell type may be identified, and the cellular differentiation process may be optimized by identifying a minimal number of targets and a corresponding CRISPR-associated guide RNA effector sequences. As described below, selected genomic targets are exposed to Cas/guide RNA complexes and are characterized to assess progress toward differentiation into a desired cell type. In certain embodiments, cycles of exposure to selected minimum numbers of effectors can continue as necessary until an endpoint is achieved.

Methods and systems for directing cell fate include selecting a minimal number of genomic targets responsible for directing cell differentiation into a desired cell type. A minimum number of guide RNA sequences corresponding to each of the selected genes are identified. The guide RNAs form a complex with a Cas protein, and the Cas-gRNA complex is introduced into each a plurality of stem cells to promote cell differentiation to a desired cell type. Cells are then assessed to determine which of them has progressed toward target cell type. Assessment may be carried out by comparing identified traits of the targeted cells to specific traits characteristic of the differentiated cell. If a desired cell end point is not achieved in the first cycle, the cycle may be repeated with a minimal number of genes thought to be associated with the desired differentiated cell type. In some embodiments, the genes identified in the first cycle may also be identified in subsequent cycles. In other embodiments, the desired cell type may be achieved after the first cycle. In yet other embodiments, the cycle may be repeated to further enhance a phenotype of the desired cell type.

To identify genomic targets and sequences of corresponding guide RNAs, aspects of the invention include analysis of data from a plurality of data sources. Preferred data sources include, but are not limited to, publications, public data sets (e.g., gene expression data sets), cell type characterization profiles, the output from systems of algorithms, and internal data sets, including laboratory results, of, for example scRNA-seq (single-cell RNA sequencing) expression data obtained from the differentiated cells produced by methods of the invention. In other embodiments, identifying initial minimum guide RNA sequences includes (1) a literature search to identify genes suspected to be involved in differentiating cells to a specified differentiated cell type and (2) searching, such as a genomic database search (e.g., in GenBank or Ensembl), to identify suitable guide RNA targets (e.g., unique or nearly-unique 20 base stretches, adjacent to a protospacer adjacent motif, within putative promoter regions of genes identified in step 1). Methods may also include (3) analysis of the data to identify a temporal sequence of gene expression to direct cell fate specification of the desired cell type.

In another embodiment, methods of the invention may further include implementation of software/algorithms to predict the activity of different gRNA sequences within a promoter sequence. Such methods of analysis include the identification of at least one gRNA per target gene that maps to the promoter region of the gene to optimally activate the gene. If the cell type is not achieved, steps 1-3 are repeated until the cell type is achieved.

Guide RNAs target promotor regions of identified genes that are known or suspected to be involved in differentiation of a selected cell type. Preferred gRNA typically includes a targeting portion of about 20 bases that hybridizes to a complementary target in double stranded DNA (dsDNA) when that target is adjacent a short motif dubbed the protospacer-adjacent motif (PAM). Identifying a minimum number of guide RNAs may include introducing into each of a plurality of cells a Cas protein and a guide RNA complex to produce a viable cell, or progeny thereof, measuring gene expression of the target in the viable cell to identify a minimum number of guide RNAs causing optimal gene expression of the target gene. Gene expression can be analyzed by methods known in the art, e.g., RT-qPCR. In another embodiment, the minimum set of guide RNAs are identified by bioinformatics analysis of the data. The guide RNAs can be a set of one to a ten guide RNAs that can be complexed with a Cas protein and delivered to a stem cell to effectively target the gene to differentiate cells into the desired cell type. In other embodiments, an effective set of gRNAs per gene may be a pool of 4-5 gRNAs. In yet another embodiment, an effective set of gRNAs may be 2-4 gRNAs per gene.

Methods of the invention may include stem cells, which may be of any cell type, including totipotent stem cells, pluripotent stem cells, and multipotent stem cells. Preferably, embodiments may use induced pluripotent stem cells (iPS cells or iPSC), which are pluripotent stem cells generated from adult cells. Methods for generating iPS cells from adult stem cells through the introduction of iPS reprogramming factors are known in the art. The iPS cells may of any origin, for instance, human iPS cells.

In certain aspects of the invention, Cas proteins are complexed with the minimum set of guide RNAs and introduced into to stem cells to target the identified genes so as to differentiate the stem cell to a desired cell type. Proteins originally found in bacteria in association with clustered, regularly interspersed palindromic repeats (CRISPR) have been termed CRISPR-associated (Cas) proteins. Cas9 endonuclease is one example of many homologous Cas endonucleases that function as RNA-guided endonucleases. Cas endonucleases can be complexed with both a trans-activating RNA (tracrRNA) and a CRISPR-RNA (crRNA), and is guided by the crRNA to an approximately 20 base target within one strand of dsDNA that is complementary to a corresponding portion of the crRNA, after which the Cas endonuclease creates a double-stranded break in the dsDNA. Variants of Cas endonucleases in which an active site is modified by, for example, an amino acid substitution, may be catalytically inactive, or “dead” Cas (dCas) proteins and function as RNA-guided DNA-binding proteins. Cas endonucleases and dCas proteins are understood to work with tracrRNA and crRNA, or with a single guide RNA (sgRNA) oligonucleotide that includes both the tracrRNA and the crRNA portions, and, as used herein, “guide RNA” or “gRNA” includes any suitable combination of one or more RNA oligonucleotides that will form a ribonucleoprotein (RNP) complex with a Cas protein or dCas protein and guide the RNP to a target of the guide RNA. When dCas protein is linked to an effector domain and complexed with guide RNA, the complex can upregulate or downregulate transcription. In other aspects of the invention, the stem cells are provided with dCas ribonucleoproteins (RNPs) linked to effector domains that participate in transcriptional regulation. When the target of the guide RNA is within a promoter, the linked effector domain can recruit RNA polymerase or other transcription factors that ultimately recruit the RNA polymerase, which RNA polymerase then transcribes the downstream gene into a primary transcript such as a messenger RNA (mRNA).

The guide RNAs (gRNAs) identified by methods of the invention are thus complexed with Cas proteins and guide the Cas proteins to their respective genomic targets within the stem cells. The gRNAs and associated Cas protein link to domains of genes identified by methods of the invention as a minimum gene necessary to differentiate the cells into the desired cell type. The methods of differentiating a cell to a desired cell type, or subtype using Cas proteins and minimum guide RNAs that target the Cas proteins to the identified minimum target genes to participate in transcriptional regulation of the cell to the desired cell type, or subtype. In yet another embodiment of the invention, the Cas protein, is a dCas protein and is linked to an effector, for example, a transcription regulator.

The complexes introduced can have various activities in the stem cells to cause cell differentiation into a desired cell type. For example, the activity may activate or repress genes that encode proteins involved in cell differentiation or may recruit coactivator or corepressor proteins to the complex to cause an activating or inhibiting activity. The desired cell type may be any cell type or subtype and may have a specific phenotype, be at any stage of maturity or state of differentiation. For example, the desired cell type may be an adult cell, an intermediary cell, an immature cell, or any cell type in between. The desired cell type may be for an external layer of the body, such as a skin cell. Alternatively, the desired cell type may be an adult cell of an internal layer of the body, such as a lung cell, a thyroid cell, or a pancreatic cell. Further, the desired cell type may be an adult cell of a middle layer of the body, such as a cardiac muscle cell, a skeletal muscle cell, a smooth muscle cell in the gut, a tubule cell in the kidney, or a red blood cell. Furthermore, the desired cell type may be an adult cell of the nervous system. In other embodiments, the desired cell type may be a target phenotype. For example, the target phenotype may be a dopaminergic neuron. In any event, the targets involved in causing a stem cell to differentiate into a specialized cell type should be known and their interactions understood.

In some embodiments, methods include identifying a temporal sequence of gene expression to differentiate the cells to the cell type. gRNAs (with or without the dCas protein linked to the regulator) may be introduced into at least one of the plurality of cells in a temporal sequence. The temporal sequence may include the introduction of a first set of one or more guide RNAs during a first period comprising one or more hours or days, followed by introduction a second set of one or more guide RNAs during a second period comprising one or more hours or days. Optionally, the first set of one or more guide RNAs and the second set of one or more guide RNAs comprise wholly different guide RNAs and/or the first period and the second period do or do not overlap in time. In some embodiments, CRISPRa/i is used against a first set of targets during the first period, the first period comprising at least two days, and using CRISPRa/i against a second set of targets during the second period to differentiate the one of the plurality of stem cells into the desired cell type. In a preferred embodiment, the desired cell type is a dopaminergic neuron.

Aspects of the invention may include identifying the cell type of the differentiated cells. Cell types are identified by specific cell traits that have been previously identified as characteristic of a certain cell type. Cell traits may include cell morphology, chromosome analysis, DNA analysis, protein expression, RNA expression, enzyme activity, cell-surface markers, or a combination thereof. Each of the differentiated cells produced by methods of the invention may be characterized by cell traits. Characterizing the cells may include identifying cell traits by staining the cells with a marker for the desired characteristic, and sorting the cells using, for example, a fluorescence-activated cell sorting instrument, a magnetic bead-based purification, others, or a combination thereof. In another embodiment, characterizing the cells may include identifying cell traits by measuring gene expression in the cell or progeny thereof. Gene expression includes one or more of: quantifying expression levels via RNA-Sequencing; measuring gene expression via single-cell RNA sequencing; or evaluating DNA-protein interaction via chromatin immunoprecipitation and DNA sequencing (ChIP-seq). The methods may include determining fold-change in expression level of a transcript associated with a marker of a specific cell type by normalizing read counts from the measuring against control read counts. The methods may also include comparing transcriptomes of individual cells to assess transcriptional similarities and differences between the cells. The cell type of each of the cells may be determined by comparing the identified traits of each of the cells to the known traits of a cell type. The methods may also include identifying cell type by comparing transcriptomes of the cells to assess transcriptional similarities and differences between the cells and may include clustering like cells. In an embodiment, the desired trait includes a specified differentiated cell type and the marker includes a protein expressed by the differentiated cell type. In another embodiment, the desired trait may be a neuronal phenotype, and marker one or more of the presence of beta III tubulin and DAPI and the absence of Oct4. In a preferred embodiment, the desired trait may include an inducible neuron phenotype, and the marker the presence of beta III tubulin.

Aspects of the invention provide systems and methods to collect, analyze, and store data sets to provide a user with cell type data. The cell type data may be any type of data described herein, for example genes involved in differentiation of a cell type and their respective genetic sequences, guide RNA sequences, lineage trajectories, genetic regulatory networks, cell line pseudo-timelines, and temporal sequences of gene expression. In an embodiment, methods and systems of the invention continue to identify genes and corresponding guide RNA sequences involved in cell fate specification of a cell type, or an enhanced phenotype of a cell type. In a preferred embodiment, the genes are the minimal genes and the guide RNAs are the minimum effective set. In another embodiment, a collection of genes (i.e., a gene module) associated with affecting a particular phenotype may be identified. In an embodiment, the gene module may also include the temporal sequence of expression of the genes of the module. The module may be utilized to obtain the phenotype in any cell type.

Multiple approaches may be employed to identify one or more genes involved in directing cell differentiation, thereby engineering cell fate. For example, machine learning may be used to predict genes or genetic regulatory networks whose alteration activates, represses, or modifies transcriptional networks to produce target cell types. When machine learning is applied, training data may include data from the database or any other source of data representing various stages of the natural development of the starting cells to the mature cell type of a cell line. Moreover, training the machine learning algorithm may include providing data from a plurality of sources (a training data set) to the machine learning algorithm and optimizing parameters of the machine learning algorithm until the machine learning algorithm produces output describing the minimal genes, the temporal sequence, and the sequences of the minimum guide RNAs to achieve a cell type.

As such, applications and methods of the invention may also include a computer-implemented method, e.g., utilizes a computer system that includes a processor and a computer-readable storage medium. The processor of the computer system executes instructions obtained from the computer readable storage device to perform the analysis receiving data from a plurality of sources to identify, for example, the minimal gene targets to differentiate a cell to a desired cell type. For example, applications of the present disclosure relate to advanced analytics (such as machine-learning) tools, systems and methods for processing data from a database, or a multitude of databases, and provides an adaptive learning processor. The disclosed processor is configured to update and optimize its logic in response to receiving electronic data from multiple sources, for example, genetic databases, user input, and experimental data related to the effectiveness of the identified gRNA on targeting the gene for optimal gene expression, or the effectiveness of the identified genes to differentiate a cell.

Advantageously, embodiments of the present disclosure provide a self-learning processor that is capable of performing adaptive learning to optimize future prediction of, for example, the effectiveness of gene targets and of different gRNA sequences. Accordingly, the disclosed system provides increasingly accurate and valuable results that allow for optimized gene targets, optimized gRNAs, and optimized temporal gene expression sequences to differentiate a cell and ultimately direct cell fate specification. Cell fate specification can be that of any cell type (or subtype) within a cell line.

Certain embodiments include a combinatorial approach in which CRISPRa/i regulates expression of some factors in combination with the direct introduction or otherwise induced expression of other factors. Methods may include initiating expression of, or introducing, additional gene products identified by methods of the present invention as being necessary to promote differentiation of the one of the plurality of stem cells into the viable cell or progeny thereof. Expression of at least one of the additional gene products may be initiated by introducing a corresponding gene using, e.g., a PiggyBac transposon; introducing a corresponding gene via a plasmid or viral vector; or introducing an mRNA encoding the gene product. The additional gene products may be introduced as a protein to the one of the plurality of stem cells. The vector may include a viral vector, a plasmid, or transposable element. Optionally, the vector further has a selection marker, and the method includes selecting for cells transformed by the vector prior to an isolating step. The cells may be selected for transformation by the vector prior to introducing the one or more guide RNAs. In an illustrative embodiment, the gene product is a transcription factor and the transcription regulator under guidance of the dCas protein and the corresponding guide RNAs results in differentiation of the one of the plurality of stem cells into a neuron. In another embodiment, the one of the plurality of stems cells may be differentiated into a dopaminergic neuron.

The disclosed systems and methods allow for the identification and characterization of targets involved in cell differentiation. These methods may be used to identify targets that can be activated, inhibited, or altered to produce cells of any target phenotype from any starting cell type. Through the application of the disclosed methods, stem cells can be transformed into specific cell types that may serve as, or may produce, useful therapeutic agents for the treatment of various diseases. As such, stem cell treatments will benefit from the ability to intentionally and efficiently direct cell fate—the inducement of stem cells to differentiate into the desired target phenotype. Additionally, methods of the disclosure may be useful for the production of artificial cells with synthetic but desired phenotypes.

In certain embodiments, the invention is directed to systems for image-driven cell manufacturing. The systems comprise a substrate having a surface on which cells are disposed; an imaging system, or imager, configured to image cells on the substrate at high resolution; and a pulsed laser scanner that directs laser pulses to the substrate under the cells.

Some embodiments further comprise a computing system comprising a processor and a computer-readable storage device. The computer-readable storage device contains instructions that when executed by the processor cause the system to: receive data from plurality of sources; perform an analysis on the data to identify targets related to cell differentiation of a desired cell type; and direct laser pulses from the pulsed laser scanner to the substrate under the targets. The imager and the pulsed laser scanner are communicatively coupled to the processor.

In some embodiments, the substrate is coated with an absorbing layer that absorbs laser pulses. In some embodiments, the substrate comprises an absorbing layer that absorbs laser pulses, the absorbing layer disposed between the surface on which cells are disposed and the pulsed laser scanner. In some embodiments, the absorbing layer is a partially absorbing layer. In some embodiments, the absorbing layer absorbs transmission at one or more imaging wavelengths in a range of about 400 nm to about 700 nm. In some embodiments, the substrate comprises titanium, gold, or a combination thereof. In some embodiments, the substrate comprises an antireflection layer. In some embodiments, the substrate comprises an antireflection layer on a surface of the substrate opposite the surface on which cells are disposed. For example, the antireflection layer may be disposed on the same side as the laser source. Therefore, when viewed in a direction moving away from the laser source and imaging side, the stacked layers of the substrate comprise an antireflection coating, a bulk substrate material (i.e., glass), a partially-absorbing layer (i.e., Ti), extracellular matrix, and cells. In some embodiments, the substrate does not leach or ablate into cell culture.

In some embodiments, the pulsed laser scanner transmits laser pulses at a wavelength of greater than about 500 nm. In preferred embodiments, the pulsed laser scanner transmits laser pulses at a wavelength of about 532 nm. In some embodiments, the pulsed laser scanner has a pulse frequency of greater than about 10 kHz. In some embodiments, the targets are mammalian genes. In some embodiments, the mammalian genes correspond to a species selected from mouse, human, and a combination thereof.

In certain embodiments, the invention is directed to methods for image-driven cell manufacturing. Methods comprises disposing cells on a substrate; imaging cells on the substrate at high resolution to create images; computing cell characteristics from the images to identify selected cells; and directing a pulsed laser scanner to deliver laser pulses to the substrate under the selected cells, thereby manufacturing cells.

In some embodiments, a coating layer of the substrate absorbs, or partially absorbs, laser pulses to convert optical energy into microbubble formation. The method further comprises destroying selected cells with the microbubble formation. The method further comprises removing selected cells with the microbubble formation. The method further comprises porating selected cells with the microbubble formation. In some embodiments, porating selected cells is a temporary poration.

In certain embodiments, the method further comprises delivering barcodes to the cells during the temporary poration, wherein the barcodes are based on cell characteristics from the cell images. The method further comprises distinguishing barcoded cells in downstream analysis.

In certain embodiments, the method further comprises introducing biological cargo to the selected cells during the temporary poration. The method further comprises introducing fluorescent indicator molecules with the biological cargo. The method further comprises imaging the selected cells for presence of the fluorescent indicator molecules, thereby verifying delivery of the biological cargo to the selected cells.

In some embodiments, computing cell characteristics comprises performing an analysis on data received from a plurality of sources to identify selected cells related to cell differentiation of a desired cell type. In some embodiments, computing cell characteristics comprises determining a minimum number of genes required for differentiation of a stem cell into a selected cell type; exposing said stem cell to a Cas endonuclease and associated guide RNAs directed at a portion of said genes; and identifying members of said selected cell type. In some embodiments, laser pulses are delivered to isolate the members. In some embodiments, the members are identified by comparing cell traits of the members to the specific cell traits of the cell type.

### BRIEF DESCRIPTION OF THE DRAWINGS

FIG. 1 shows a flowchart of a workflow of the invention.

FIG. 2 shows an exemplary method of the invention.

FIG. 3 shows a flowchart of a workflow of the invention.

FIG. 4 shows an exemplary method of the invention.

FIG. 5 shows a flowchart of a workflow of the invention.

FIG. 6 shows an exemplary method of the invention.

FIG. 7 shows a flowchart of a workflow of the invention.

FIG. 8 shows an exemplary method of the invention.

FIG. 9 shows an exemplary method of the invention.

FIG. 10 shows an exemplary method of the invention.

FIG. 11 shows an exemplary method of the invention.

FIG. 12 shows a flowchart of a workflow of the invention.

FIG. 13 shows an exemplary method of the invention.

FIG. 14 shows a flowchart of a workflow of the invention.

FIG. 15 shows a flowchart of a workflow of the invention.

FIG. 16 shows an exemplary method of the invention.

FIG. 17 shows a flowchart of a workflow of the invention.

FIG. 18 shows an exemplary method of the invention.

FIG. 19 shows an exemplary system according to the invention.

FIG. 20 depicts bar graph of an exemplary RT-qPCR of CRISPRa activation of target genes in iPSCs using gRNA sequences predicted by methods of the invention.

FIG. 21 presents a timeline of initial inducible neuron induction as an example.

FIG. 22 depicts a bar graph of exemplary RT-qPCR data and exemplary immune-fluorescence images of day three (3) cell differentiation of neurons.

FIG. 23 depicts a bar graph of exemplary RT-qPCR data of day seven (7) cell differentiation of neurons.

FIG. 24 depicts exemplary immune-fluorescence images.

FIG. 25 illustrates an exemplary t-SNE graph depicting a cell clustering analysis using single-cell RNA sequencing data from day ten (10) cell differentiation neurons.

FIG. 26 illustrates exemplary immune-fluorescence images.

FIG. 27 depicts a bar graph of the neuron GRN status of the nine (9) clusters of FIG. 25.

FIG. 28 depicts four (4) t-SNE plots mapping genes and the nine clusters of cells to show the gene expression of those genes in those clusters of cells.

FIG. 29 depicts a graph of the classification of neuronal subtypes (x-axis) and the percentage of cells in each cluster (y-axis).

FIG. 30 illustrates an exemplary t-SNE graph depicting a cell clustering analysis using single-cell RNA sequencing data from the developing human midbrain.

FIG. 31 illustrates an exemplary t-SNE graph depicting cell subtype clusters using previously established gene signatures of neural subtypes.

FIG. 32 is a graphical representation of the gene enrichment (y-axis) of the identified subtypes (x-axis) in FIG. 31.

FIG. 33 shows a model depicting differentiation trajectories of neural subtypes.

FIG. 34 depicts four (4) t-SNE plots mapping genes and the different subtypes of cells to show the gene expression of those genes in those subtypes of cells.

FIG. 35 depicts three the top 13 up-regulated and top 13 down-regulated genes responsible for establishing the GRNs responsible for each cellular subtype identity as ranked by their respective GRN score.

FIG. 36 shows the exemplary mapping of the top level genes of the gene regulatory networks of the differentiation pathways from a neural progenitor cell to hDA1 and hDA2 subtypes.

FIG. 37 illustrates the predicted relative expression levels of the top level genes associated with mature dopaminergic neurons plotted across time (right), and the derivate of these expression levels (left) identifies inflection points in gene expression.

FIG. 38 provides the results of the CellNet analysis of the predicted manipulation of GRNs and resulting verification of cell line.

FIG. 39 provides the predicted gene regulation analysis of MYT1L and BASP3 during hDA2 differentiation.

FIG. 40 depicts a bar graph of the GRN status over time of NPCs and neurons during differentiation.

FIG. 41 illustrates a detailed block diagram of electrical systems of an example computing device in accordance with an example embodiment of the present disclosure.

FIG. 42 shows a system to identify the minimum number of targets to specify cell fate.

### DETAILED DESCRIPTION

Methods and workflows for image-based laser-scanning of laser-activated constructs for various applications involving cells are described. The use of laser-activated constructs, light absorbers capable of absorbing laser radiation and transferring the energy to the environment, for intracellular delivery, extraction of cargoes from within cells, and cell killing, cell detachment, or a combination thereof are described. The invention uses various methods for intracellular delivery techniques for large molecules and particles. Using information acquired from images enables the laser-scanning parameters for delivery, extraction, killing, cell detachment, or a combination thereof to be optimized depending on the state of the cells and the intended application.

Examples of intracellular delivery techniques are described in U.S. Provisional Application No. 62/570,149, U.S. Provisional Application No. 62/686,109, U.S. Provisional Application No. 62/686,120, and U.S. Provisional Application No. 62/701,863, the contents of each of which is incorporated herein in its entirety by reference.

In image analysis in conjunction with laser-based poration, a pulsed laser beam is focused to a small volume in close proximity to a cell membrane, resulting in plasma and/or bubble formation in the aqueous environment. Such a technique is described in U.S. Pat. No. 5,795,755, which is hereby incorporated in its entirety by reference. The bubble's expansion and collapse generates a shockwave that is capable of generating temporary pores in nearby cells. However, this technique suffers from several disadvantages, including low throughput, due in part to the fact that the laser beam is focused in a small volume and therefore only cells within a small region in close proximity are permeabilized.

An alternative, higher-throughput, technique for cell poration involves the use of laser-activated constructs capable of absorbing laser radiation. Upon laser illumination, the laser activated construct absorbs energy, transferring the energy to the environment to generate a bubble. The bubble expands and collapses, generating a shockwave that temporarily permeabilizes the membranes of cells in close proximity. Further, laser-based cell poration techniques are described in the context of delivering membrane-impermeable cargoes into cells in Courvoisier et al., 2015, Nano Letters, Plasmonic Tipless Pyramids for Cell Poration; Saklayen et al., 2017, ACS Nano, Intracellular delivery using nanosecond-laser excitation of large-area plasmonic substrates; Chen et al., 2017, Applied Physics Letters, Dynamics of transient microbubbles generated by fs-laser irradiation of plasmonic micropyramids; International Publication No. WO 2016/127069 A1; and US Patent Publication No. 2012/0171746 A1, the content of each of which is incorporated herein by reference.

The present invention is concerned with applications involving the use of laser-activated constructs to transfer energy to cells, for the purposes of intracellular delivery of cargoes, extraction of cargoes from within cells, and cell killing and/or detachment. The present text is concerned primarily with applications in which information regarding the cells (including, but not limited to: cell density, cell type, cell morphology, intracellular protein expression, surface protein expression, etc.) is useful in determining the optimal laser irradiation parameters (including, but not limited to: illuminated area, laser fluence, number of laser pulses, timing between laser pulses, speed of laser scan, number of laser scans, timing between laser scans).

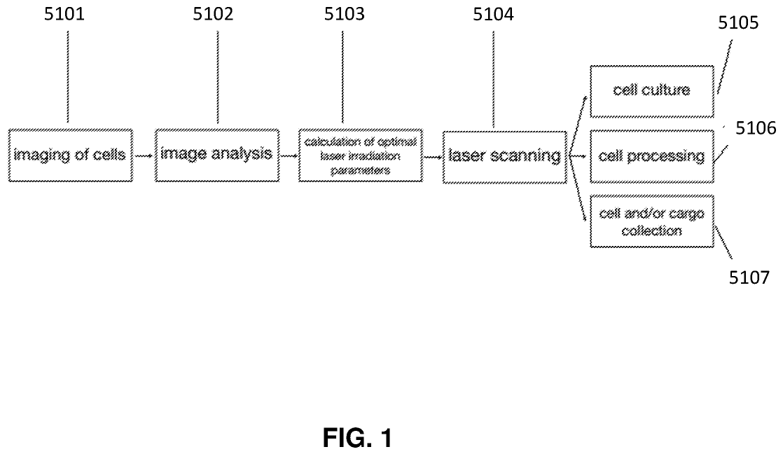

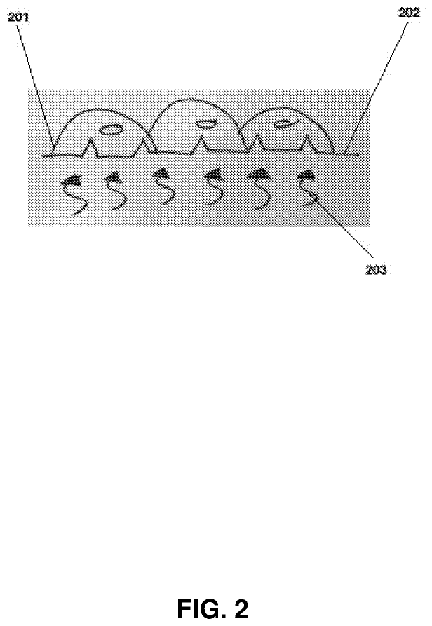

FIG. 1 shows a flowchart of a representative workflow. Imaging of cells 5101 may take place while cells are in contact with the laser-activated construct, as shown in FIG. 2, in the case where cells 201 are cultured directly on the laser-activated surface 202. In other embodiments, during imaging the cells may be resting on a commonly used cell culture surface, such as glass, polystyrene, or polystyrene coated with a biological matrix. In this case the laser-activated constructs may be added to the cell media and allowed to settle onto the cells via gravity or centrifugation, or a surface containing laser-activated constructs may be lowered onto the cells from above. Depending on the information needed, the cells and various components of the cells (including but not limited to: organelles, membranes, intracellular proteins, surface proteins, and other markers) may be stained prior to imaging. Imaging may involve the use of brightfield microscopy, phase-contrast microscopy, fluorescence microscopy, confocal microscopy, and/or spectroscopy (e.g. near-infrared spectroscopy, infrared spectroscopy, raman spectroscopy, etc.).

There is a wide range of tools available for image analysis 5102, and the appropriate tool and analysis will depend on the information needed. For example, if information regarding cell density is required, a brightfield, phase-contrast, or fluorescence microscopy image could be opened in ImageJ or FIJI and a plug-in or script could be used to count the number of cells in a given area. An example of such a cell counting script is given in Schindelin et al., 2012, Nature Methods, Fiji: an open-source platform for biological-image analysis, which is hereby incorporated in its entirety by reference. Similarly, image analysis may be used to characterize cell morphology (shape, volume, etc.), protein expression (both intracellular and surface proteins), and cell type (based on morphology, protein expression, etc.).

The information gathered from the image analysis can be used to determine the optimal parameters for laser irradiation 5103 of the laser-activated constructs, whether the application is intracellular delivery, extraction of cargo from within cells, killing of cells, and/or detaching of cells (dead or alive). Laser irradiation 5104 may be done at various levels of resolution—for instance, an entire sample/well may be irradiated with a given set of laser parameters, a subpopulation of cells within a sample/well may be irradiated with a given set of laser parameters, a single cell within a sample/well may be irradiated with a given set of laser parameters, or a subregion of a single cell may be irradiated with a given set of laser parameters. The laser parameters that can be varied based on the image analysis may include but are not limited to: area of illumination, laser fluence, number of laser pulses (in the case of a pulsed laser system), timing between laser pulses, speed of laser scanning, number of laser scans, and/or timing between laser scans.

After laser irradiation, the cells may be placed back into culture 5105, the cells and/or cargoes may be collected for later use 5107 (for instance, if proteins are being extracted and collected from cells for therapeutic applications), or the cells may be further processed before collection (for instance, if cells must be washed and/or stained for downstream analysis) 5106.

FIG. 2 shows methods of the invention. Cells 201 may be cultured in close or direct contact with a laser-activated construct 202. The cells may also be cultured on a commonly used cell culture surface, such as polystyrene, which may be coated with a biological matrix such as laminin, Matrigel, Geltrex, Cultrex, or collagen, etc. A laser-activated construct is used to absorb laser radiation 203 and transfer the energy to the surroundings, and ultimately, to the cell membrane. Examples of such constructs include but are not limited to: a patterned surface, with which cells may be in contact; a patterned surface, from which cells may be a set distance away; or nanoparticles, of any shape, that are cultured with the cells or allowed to come into close contact with the cells via gravity or centrifugation. The constructs used to absorb laser irradiation may be composed of and/or coated in metal or any light-absorbing material. The primary requirement for such constructs is the ability to absorb laser radiation and transfer said energy to the environment. The results of the energy transfer to the environment may include but are not limited to: increase in temperature, shockwave formation, bubble formation, and/or plasma generation.

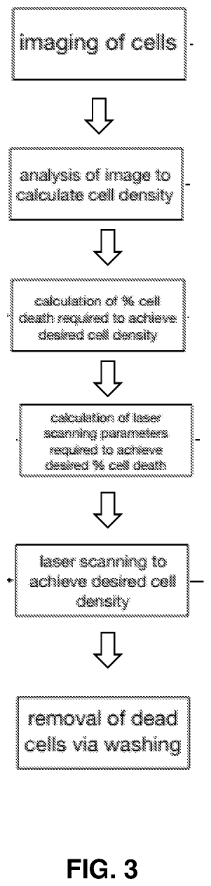

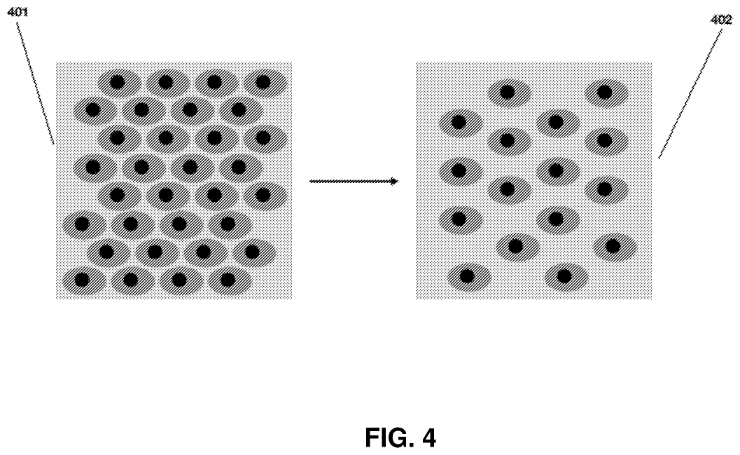

FIG. 3 shows a flowchart of a workflow. In one embodiment of the image-based laser scanning technique described here, the technique may be used to uniformly kill/detach cells to achieve uniformly confluent cells of varying cell densities. The initial cell density at the time of imaging 401 may be within the range of 0-100% of the surface area being covered by cells, and the resulting cell density after laser scanning 402 may be within the range of 0-100% of the surface area being covered by cells (FIG. 4).

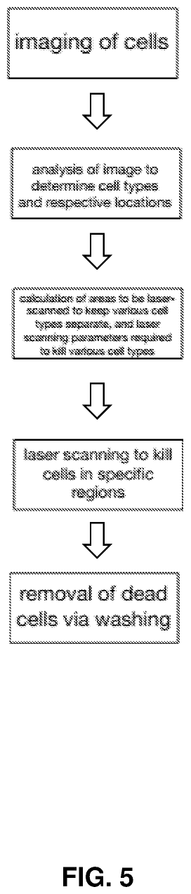

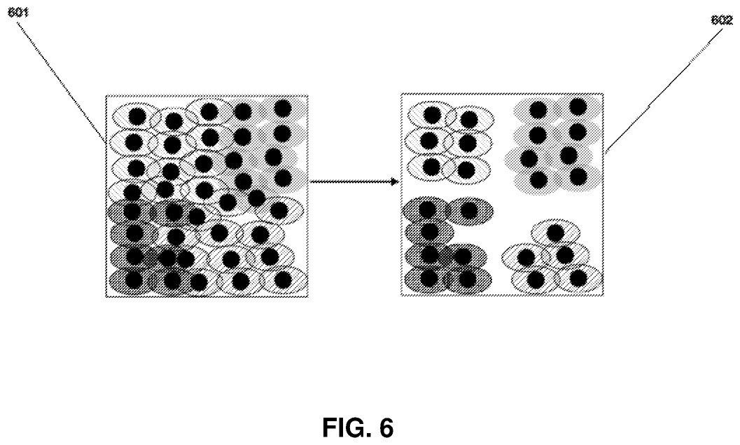

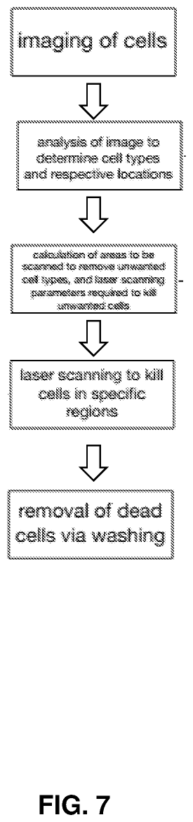

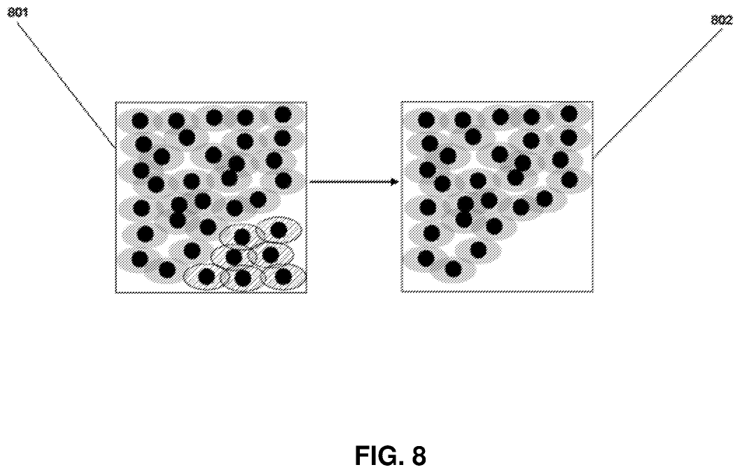

In another embodiment, the image-based laser scanning technique may be used to selectively kill cells within specific regions of a sample or cell population. This may have many applications, including but not limited to: patterning of cells, cell sorting via elimination of cells not expressing the desired phenotype, and maintaining separation between multiple populations of cells within a single sample. The workflow in FIG. 5 depicts how image-based laser-scanning could be used to keep various populations or colonies of cells separate within a single sample, well, or cell culture dish. At the time of imaging, multiple subpopulations of cells may be overlapping or growing in close proximity to each other 601; after laser-scanning, the subpopulations are separated 601 (FIG. 6). The workflow in FIG. 7 depicts how image-based laser-scanning could be used as a cell sorter to remove unwanted cells (for instance, cells that are expressing an undesired phenotype) from the population. At the time of imaging, the unwanted cells are present in the population 801; laser-scanning is then performed to kill and/or detach the unwanted cell types 802 (FIG. 8).

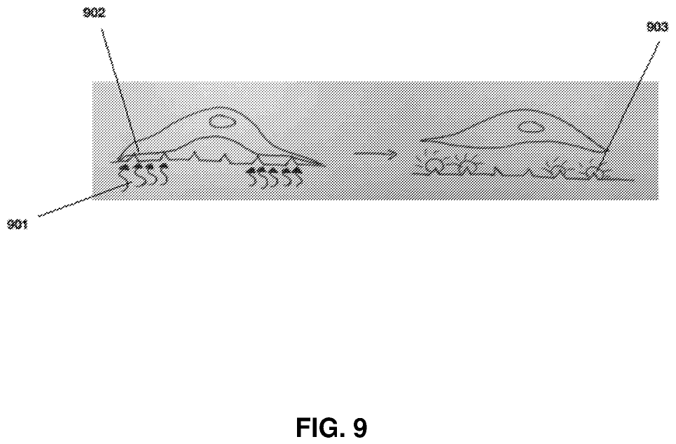

The image-based laser-scanning technique described here may be used to selectively detach cells of interest within a population, with or without killing the cells in the process. Cavitation bubbles and their accompanying shockwaves have previously been used to detach cells from substrates, as described in Ohl and Wolfrum, 2003, Biochimica et Biophysica Acta, Detachment and sonoporation of adherent HeLa-cells by shock wave-induced cavitation, which is hereby incorporated in its entirety by reference. However, the image-based laser-scanning technique described here is unique in that it offers control over which specific cells are being detached. For instance, if there are cells within a population that are expressing a specific phenotype of interest, those cells may be selectively detached and collected for further processing. Unlike the cavitation-based technique, the laser-based technique described here enables precision at various levels, including cellular and subcellular. In certain applications it may be advantageous to laser-illuminate 901 only the areas where the cell's filopodia 902 are in contact with the laser-activated constructs (FIG. 9). This would result in fewer perturbative events 903 (i.e. bubbles, shockwaves, etc.) experienced by the cell, which may provide a gentler way of detaching cells from the substrate, thus improving the viability of the detached cells.

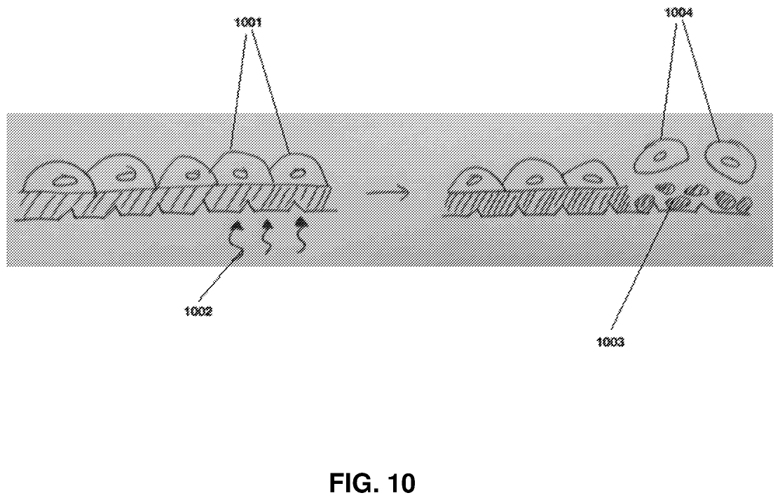

In another embodiment, the image-based laser-scanning technique described here may be used to selectively detach cells of interest within a population through the use of a layered construct between the laser-activated construct and the cell or cells of interest. The layered construct may be composed of a matrix, gel, or polymer. Cells may be cultured directly on top of the layer if the layer is composed of a biocompatible material, or the layer may be coated in a biocompatible material such as a biological matrix to promote cell adhesion and viability. The layer may have adhesive or cohesive properties that are temperature- and/or pressure-sensitive and enable it to detach or dissociate in response to perturbative events generated by the laser-activated construct upon laser illumination. For instance, some gels dissolve in response to increased temperatures, as described in Petka et al., 1998, Science, Reversible Hydrogels from Self-Assembling Artificial Proteins, which is hereby incorporated in its entirety by reference. In an embodiment using such a gel, only the region of the laser-activated construct in proximity to the cells of interest 1001 would be laser-illuminated 1002 (FIG. 10). The resulting increase in temperature would dissociate the temperature-sensitive gel 1003, selectively releasing the cells of interest 1004 and allowing them to be collected for further processing.

In another embodiment, the light source used for imaging may be the same light source used to activate the laser-activated surface. For instance, the same laser used for cell killing/detachment may be used to generate a map of the location and density of the cells cultured on the laser-activated surface. In such an embodiment, a laser with a sufficiently small spot size (exact size dependent on cell type) would be scanned across the cell culture surface, and a detector could be used to measure the scattered light or drop in transmitted light due to the presence of cells.

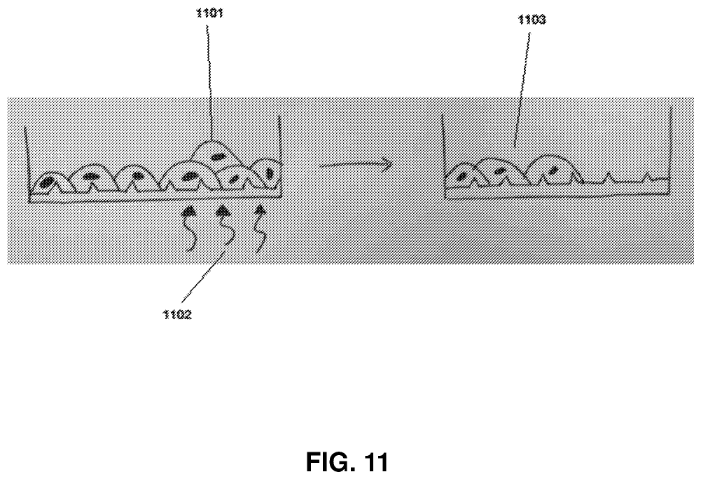

In another embodiment, the technique described here may make use of images of three-dimensional (3D) cell cultures to determine appropriate laser-scanning parameters. One potential application for such an embodiment is the use of laser-scanning to maintain monolayers of cells by detaching and/or killing cells that have begun to grow on top of each other and form 3D colonies. In such an embodiment, the 3D cell culture could be imaged using confocal microscopy, multiphoton microscopy, or optical coherence tomography, as described in Graf and Boppart, 2013, Methods Mol. Bio., Imaging and Analysis of Three-Dimensional Cell Culture Models, which is hereby incorporated in its entirety by reference. The resulting image would be analyzed to determine the regions where cells form a 3D colony 1101 (FIG. 11), and the laser could then be scanned in those regions 1102 at a fluence sufficient to detach and/or kill those cells. At the time of imaging, cells would be present both in monolayers and layered 3D colonies 1101; after laser-scanning, only monolayers of cells would remain 1103.

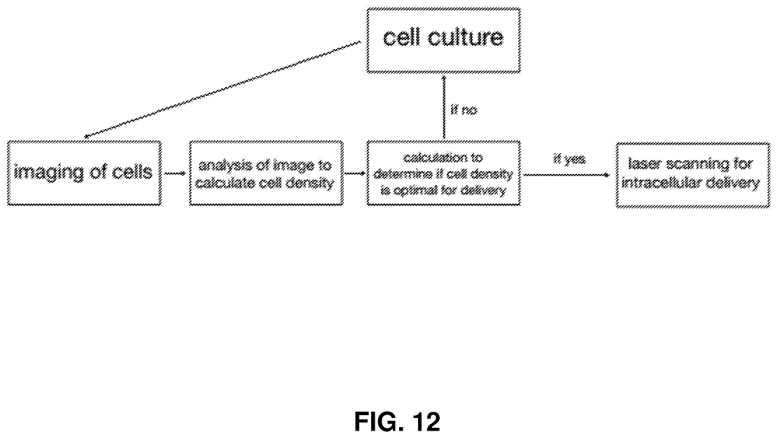

In another embodiment, the technique described here may be used to determine the optimal parameters and timing for laser scanning for intracellular delivery based off of information acquired during imaging. Information that can be acquired during imaging and may affect the optimal laser parameters and timing for cell poration and intracellular delivery include but are not limited to: cell density, cell morphology (shape, volume, etc.), and cell type (morphology, protein expression). The workflow shown in FIG. 12 depicts how cell density information acquired from imaging at multiple timepoints may be used as a parameter to determine the optimal time to laser-scan for intracellular delivery.

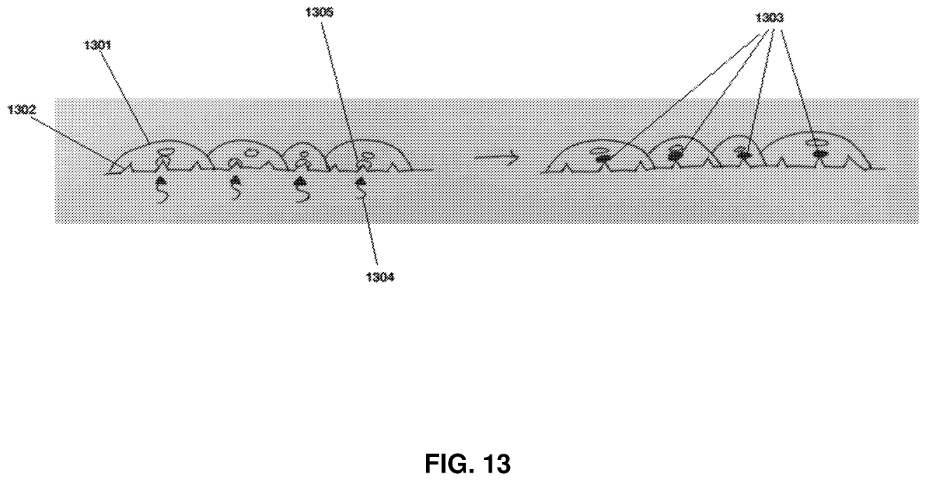

The laser-scanning technique described here enables precision at the population, cell, and subcellular level. It is therefore also possible to optimize laser scanning parameters for cell poration for intracellular delivery to each cell based on individual cell characteristics. For instance, various cells in a population may be in contact with a different number of laser-activated constructs, and illumination of each laser-activated construct would correspond to the generation of a single pore, resulting in a different number of pores generated per cell. However, it may be advantageous to generate the same number of pores in each cell. In this case, imaging may be used to determine the number of laser-activated constructs in proximity to each cell, and to pattern the laser-scanning appropriately to ensure that the same number of laser-activated constructs is illuminated per cell, thus generating the same number of pores in each cell. FIG. 13 depicts a population of cells in which each cell 1301 is in contact with a different number of laser-activated constructs 1302. To ensure that the same number of pores 1303 (in this example, a single pore per cell) are generated in each cell, the same number of laser-activated constructs are illuminated 1304 per cell. This results in the same number of pore-forming perturbative events (i.e. bubble or shockwave formation 1305) per cell.

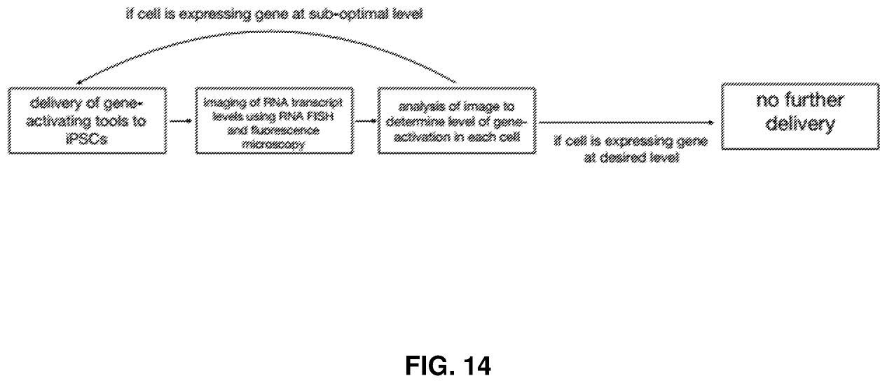

In another embodiment, the image-based laser-scanning technique described here may be used to optimize subsequent intracellular delivery experiments based on imaging results acquired from initial delivery experiments. Information that can be acquired during imaging of initial intracellular delivery results and may affect subsequent delivery experiments include but are not limited to: cell density, cell morphology (shape, volume, etc.), and cell type (morphology, gene expression, etc.). For instance, guide RNAs and CRISPRa ribonucleoprotein complexes (RNPs) can be delivered to induced pluripotent stem cells (iPSCs) to activate particular genes. In one example, RNA FISH (fluorescent in situ hybridization targeting ribonucleic acid molecules) may be used in combination with fluorescence microscopy to quantify the levels of mRNA transcripts (i.e. gene activation) present in each cell. Analysis of the fluorescence microscopy images can be used to determine whether the desired gene has been activated at an optimal or suboptimal level in each cell of the cell population. Laser-scanning may then be used to re-deliver guide RNAs and CRISPRa RNPs specifically to the cells expressing the desired gene at a suboptimal level, in order to improve gene activation levels. An example of this workflow is depicted in FIG. 14.

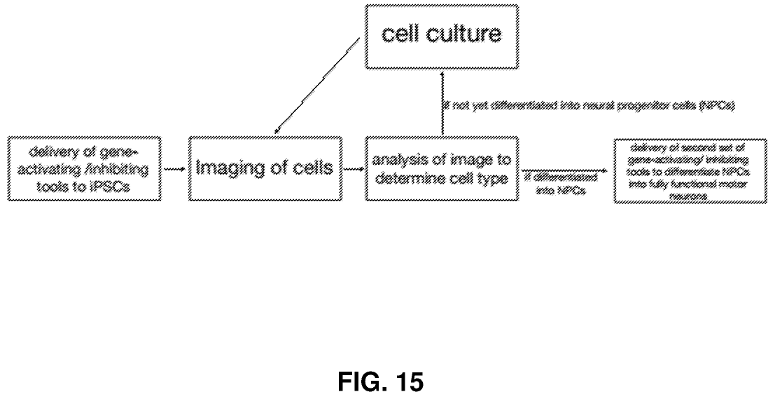

The image-based laser-scanning intracellular delivery technique described here may also make use of a time series of images to optimize laser-activated delivery parameters at multiple timepoints. For instance, in one application, cargoes may be delivered to iPSCs at multiple timepoints to cause them to first differentiate into neural progenitor cells (NPCs), and to then differentiate into fully functional electrically-active motor neurons. As in previously described potential embodiments, iPSCs would be cultured on a laser-activated surface. For the first round of delivery, as previously described here, information acquired from the images regarding cell density and the number of laser-activated hotspots per cell could be used to determine the optimal laser-scanning parameters for delivery. These laser-scanning parameters would then be used to deliver the first set of cargoes for differentiating the iPSCs into NPCs. The potential cargoes that could be delivered for the purpose of differentiating cells include but are not limited to: CRISPRa RNPs and guide RNAs, CRISPRi RNPs and guide RNAs, episomal vectors, mRNAs, siRNAs, and transcription factors. Imaging of these cells at multiple timepoints after laser-activated delivery could be used to determine, based on cell morphology and gene expression (gene expression can be determined via methods such as fluorescence microscopy imaging in combination with RNA FISH, immunofluorescence staining, etc.), whether the iPSCs have differentiated into NPCs. Once the iPSCs have differentiated into NPCs, a second round of laser-activated delivery could be used to deliver cargoes to cause the NPCs to differentiate into fully functional motor neurons. An example of this workflow is depicted in FIG. 15.

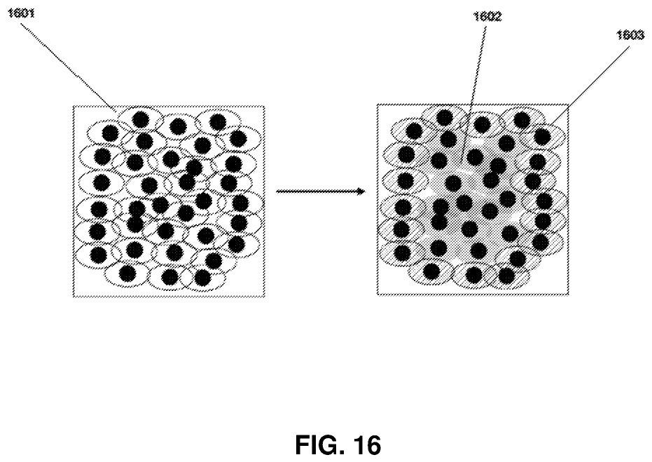

In another embodiment, the technique described here may be used to spatially-selectively deliver cargoes to cells based on information acquired during imaging. This may have utility in various applications, including the generation of heterogenous tissues and/or organoids for biological and biomedical applications. For instance, it is known that cell organization (i.e. cells' spatial positions relative to each other) is an important consideration in generating heterogenous tissues and/or organoids, as described in Yin et al., 2016, Cell Stem Cell, Engineering Stem Cell Organoids, which is hereby incorporated in its entirety by reference. Cargoes such as gene activators, gene inhibitors, transcription factors, and/or gene-editing tools may be delivered to embryonic stem cells (ESCs), induced pluripotent stem cells (iPSCs), adult stem cells (ASCs), and/or other cell types to engineer the desired cell types for the generation of a heterogenous tissue and/or organoid. In one example of such an embodiment shown in FIG. 16, image-based laser-activated delivery may be used to generate a heterogenous tissue composed of two adult cell types with a particular spatial cell organization. First, imaging of iPSCs cultured on a surface 1601 would be performed to determine the cells' relative spatial positions. CRISPRa RNPs (a gene-activating tool) and guide RNAs would then be spatially-selectively delivered to particular iPSCs to induce differentiation into the first desired cell type 1602. Finally, CRISPRa RNPs and guide RNAs would be spatially-selectively delivered to a different set of particular iPSCs to induce differentiation into the second desired cell type 1603.

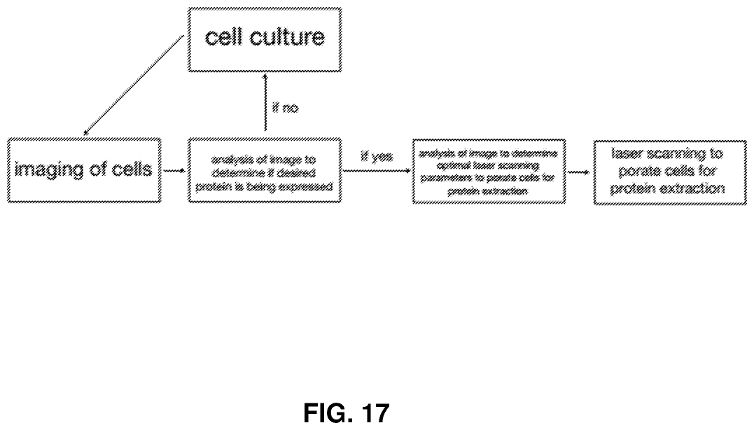

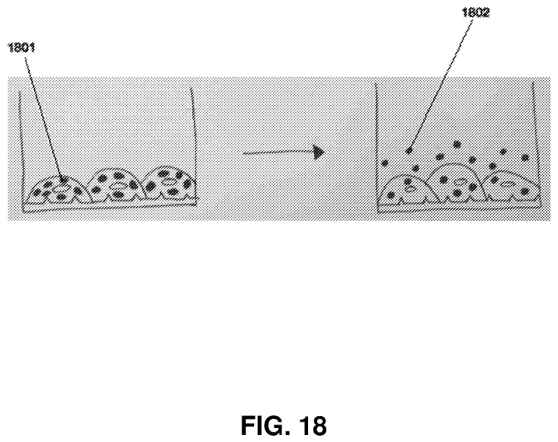

In another embodiment, the technique described here may be used to determine the optimal parameters and timing for laser scanning for extraction of cargoes from cells. Information that can be acquired during imaging and may affect the optimal laser parameters and timing for cell poration and extraction of intracellular cargoes include but are not limited to: cell density, cell morphology (shape, volume, etc.), and cell type (morphology, protein expression). For instance, imaging may be used to determine if a population of cells is expressing a particular protein of interest, and subsequent laser scanning may be used to porate the cells to extract the protein for therapeutic applications. An example of a representative workflow for this process is depicted in FIG. 17. At the time of imaging, if the cell population is determined to be expressing the protein of interest 1801 (FIG. 18), the cells are laser-scanned. The laser-scanning process porates the cells and allows the protein of interest 1802 to diffuse out of the cells into the extracellular media, where it can be extracted for further use.

Recent synthetic biology tools (CRISPRa/i, TALENs, Zinc fingers) allow for directly activating or inhibiting gene expression, and thereby skip the slow conversion from external chemical/mechanical cues through the GRNs to phenotype changes. Approaches using tools such as CRISPR reduce the “step time” in cell differentiation from weeks to days. In theory, this should allow much faster development of cell differentiation recipes/programs, but application of traditional tools has severely limited such development.

As the differentiation process is compressed from weeks to days, the control over signals has to be compressed from days to hours. Precise, discrete delivery timing results in precise timing of cross over cell membrane, and delivery of a “bare” construct that is immediately active. This has a transient effect (when desired), and the ability to deliver at multiple time points. As the differentiation process is compressed from weeks to days, the process cannot afford to disrupt cells, and cell-cell connections, by passaging the cells from substrate to substrate.

The invention provides continuous culture of adherent cells. Systems of the invention advantageously provide spatial control over delivery parameters in order to enhance efficiency of experiments. In some embodiments, systems of the invention allow for tailored, engineered populations of different cell phenotypes.

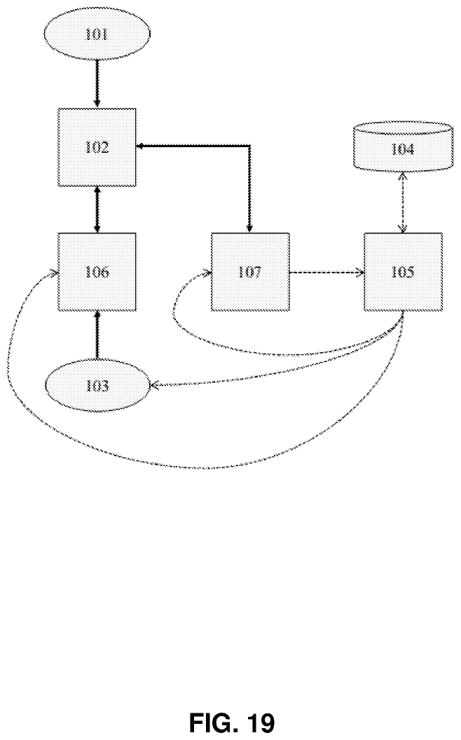

The present invention is shown in FIG. 19. Systems of the invention may be used to find the most efficacious set of gene activation and/or inhibition (modulation) steps for differentiating or transdifferentiating cells from an initial state/phenotype to one or more target states or cell phenotypes.

Cells 101 in the initial state are an input to the system. These cells are placed into a cell culture subsystem 102 which maintains cell health and is compatible with high throughput, combinatorial experiments.

A library of gene activator and/or inhibitor constructs 103 (gene modulators) is another input to the system. This may be a library that has genome-wide reach, or a subset of constructs specific to the target cell differentiation process.

A data storage subsystem 104 stores the characteristics of the target cell state/phenotype, for example RNA transcriptome data from a mature, functional cell type corresponding to the target. In addition, it may store data from experiments performed by the system, including data from cells at many intermediate states.

A computing subsystem 105 utilizes initial and target cell state data, as well as any available intermediate data, to select a series of sets of activators and inhibitors available in the library 103 based on their likelihood of routing cells from the initial or an intermediate state towards the target state. The computing subsystem may utilize cell state and route data to determine new gene modulators to add to the library 103 (ordering from vendor, or synthesizing them—longer lead time).

Gene modulators selected by the computing subsystem 105 for an experiment are pulled from the library 103 and inserted into an intracellular delivery system 106. This intracellular delivery subsystem 106 accepts cells from cell culture system 102 and delivers gene modulators, or combinations of gene modulators, from library 103 into the cells. The delivery subsystem is able to deliver single or multiple gene modulators into each cell, and to vary these modulators or combinations sample-by-sample within an experimental matrix. After delivery of gene modulators, cells are returned to cell culture subsystem 102 where cell media changes, etc. are performed to maintain cell health and allow cell differentiation.

At one or more intervals during the cell differentiation process cells are moved to an analysis subsystem 107. The analysis subsystem may assess cell health, proliferation, gene expression, functional characteristics and therefore complete or partial differentiation from the initial cell state towards the target cells type—or into unwanted cell types. Remaining may then be returned to cell culture 102 for further gene modulation deliveries and/or timepoint analyses, if desired. The computing subsystem 105 may direct the analysis subsystem to measure specific cell characteristics, markers, or expression to most efficiently determine whether cell differentiation is occurring along expected routes.

The results of the analyses by subsystem 107 are provided to the computing subsystem 105 which uses this information to build and store a revised and more complete “routing map” in storage subsystem 104.

The cell routing maps generated are in turn used to refine the combinations and timing of gene modulators in further experiments, until an efficacious routing program consisting of one or more gene modulators delivered at one or more timepoints, and resulting in a cell phenotype close to the target state, is determined. The system may in addition identify stable intermediary that are “jumping off” points for future differentiation programs, or stable cell states that correspond to non-target mature cell types, or even stable cell states that do not correspond to naturally-occurring cell types.

It may be desirable for the system to be capable of computing “programs” with multiple delivery timepoints, for example delivery of one set of gene modulators (individual modulators, or combinations of modulators, to different cells or cell samples in an experimental matrix) by delivery subsystem 106 at a first time point, and then a delivery of a second set of gene modulators (or combinations thereof) at a second timepoint, which may be hours, days, or even weeks later, according to a potential cell state trajectory (and matching “steering” gene modulation signals) computed by computing subsystem 105 according to data and partial or complete cell state and routing information accumulated in storage subsystem 104. The sequence of deliveries may be computed based on based on externally-supplied data, on experimental results produced by analysis subsystem 107 in prior experiments, or on real-time data produced by analysis subsystem 107 measuring the state of the current experiment—in other words, “real-time” (in experimental terms) adjustment of “steering” (gene modulation) signals based on observed cell state trajectories.

It may be desirable for the system, including cell culture subsystem 102 and delivery subsystem 106, to maintain cells in a continuous culture for an entire—or at least portions of—an entire experiment. For example, it may be desirable, from a cell differentiation and health standpoint—and to produce data with maximum signal to noise ratio (where “signal” is the effect on cell state/differentiation on the cells, and “noise” is cell state changes resulting from other environmental factors, including stress from cell passaging)—to maintain cells in an adherent continuous culture. Furthermore, continuous culture embodiments of the present invention may be particularly desirable because of cell-cell interactions that are often critical to differentiation pathways and cell maturation. Specifically, it may be desirable for adherent cells to rest and proliferate on a substrate (including appropriate biological matrices) within cell culture subsystem 102 for some period of time before each gene modulator delivery by delivery subsystem 106 (while in continuous adherent culture), and then be returned to cell culture subsystem 102 for another period of time to recover from such delivery and to allows unimpeded action of the gene modulators.

In some embodiments, cells will remain in adherent continuous culture for one or more such delivery cycles, and then be passaged after recovery to allow proliferation.

In some embodiments, analyses by the analysis subsystem 107 are made while cells are in continuous culture. Such analyses may include non-destructive measurements, such as imaging or label-free biochemical measurements, after which the cells are moved back to cell culture subsystem 102. Terminal testing may also be performed on adherent cells: for example, fluorescence imaging to measure cell morphology, surface markers, RNA or protein expression, etc. may provide valuable information when done on adherent cells, with knowledge of how gene modulators were delivered spatially throughout the cell culture.

For non-adherent cell types or cell cultures (where cells have transitioned from adherent to suspended in colonies), it may also be desirable to maintain these in continuous culture for the reasons outlined above, especially immediately before, during, and after delivery steps.

Delivery subsystem 106 may use a range of intracellular delivery techniques. Traditional biochemical tools for delivery of constructs into cells fall short of requirements for a high-throughput cell differentiation discovery platform. Biochemical methods have uncertain and extended timing in terms of crossing the cell membrane, as well as uncertain and extended timing in terms of being released within the cell. Therefore, biochemical methods are difficult to control from a concentration and level perspective.

Physical delivery techniques have more distinct timing, and are able to deliver bare cargos that can act immediately within the cell. Physical delivery techniques could be applied to a high throughput cell or trans-differentiation discovery system and satisfy the requirements for timing specificity, transient effect, and continuous cell culture. For example, electroporation is the use of electric fields to disrupt cellular membrane. Thermal disruption is the use of elevated temperature/temperature changes to disrupt membrane. Optoporation is the use of direct optical radiation to disrupt membrane. Cavitation is the use of ultrasound, or laser cavitation, to mechanically disrupt the membrane. Fluid shear is the use of rapid liquid flow relative to the membrane to disrupt it. Osmotic/hydrostatic treatment use concentration gradients to disrupt the membrane. Squeezing uses mechanical forces on the membrane to disrupt the membrane. Nanoneedles include the use of needles, possibly coated with cargo, that puncture the cellular membrane. Microinjection uses conventional needles to puncture the membrane. Ballistic particles use high-speed particles, possibly coated with cargo, to penetrate the membrane. Example intracellular delivery technologies and techniques are described in Stewart et al., 2018, Chem Rev, Intracellular Delivery by Membrane Disruption: Mechanisms, Strategies, and Concepts, 22; 118(16):7409-7531, which is incorporated herein by reference in its entirety.

However, physical delivery techniques have not been envisioned for use in high-throughput differentiation experiments using synthetic constructs to control gene expression. Specifically, there is a strong advantage to keeping adherent cells in continuous culture, and many conventional techniques have not been applied to adherent cell cultures. Moreover, none of these techniques have been realized in forms where they can provide both sufficiently high throughput for experiments requiring at least thousands if not hundreds of thousands or millions of cells, but at the same time provide tailored delivery depending on local cell culture conditions including cell density, cell morphology, cell phenotype or expression, etc. When cells transition through multiple states, the ability to use automatable, physical techniques to manage differentiation and proliferation is a key advantage. This, in turn, requires the ability to spatially tailor such physical delivery, detachment and/or removal techniques.

As a result, in most cases the delivery subsystem 106 will be based on a physical delivery system with precise delivery timing. In some cases, it will be capable of spatially-resolved delivery modulation, such as described in U.S. Provisional Application No. 62/717,581, the content of which is incorporated herein by reference in its entirety. In such a configuration, the analysis subsystem 107 may provide image information from which spatial delivery parameters for delivery subsystem 106 are calculated.

### Gene Modulators

A range of gene modulators may be delivered by the delivery subsystem 106 in the present invention. Ideally these modulators are capable of providing transient activation or suppression of targeted gene expression; mechanisms for achieving this include but are not limited to transcription factors, Zinc finger nuclease (ZFN)-based activators or inhibitors, TALEN-based activators or inhibitors, or CRISPR-based activators or inhibitors, for example catalytically inactive Cas (dCas) endonuclease proteins linked to effector domains that participate in transcriptional regulation (“CRISPRa” and “CRISPRi,” the activating and inhibiting forms, respectively). The use of CRISPR-type mechanisms in systems of the invention are described in U.S. Provisional Application No. 62/660,577, the content of which is incorporated herein by reference in its entirety. An example embodiment of the present invention uses induced pluripotent stem cells (iPSCs) that have CRISPRa integrated and stably expressed. A library 103 of guide RNAs that are delivered by delivery subsystem 106 is used in order to complex with the dCas9 proteins and guide them to locations in order to activate or inhibit gene expression. The two-part system allows the intracellular delivery of relatively small gRNA molecules to transiently modulate gene function, allowing high efficiency and cell viability, and thereby enabling multiple delivery cycles with a high yield of cells that have had proper gene modulation sequences.

Biological techniques useful in the invention include those described in U.S. Provisional Application No. 62/739,027, the content of which is incorporated herein by reference in its entirety, and described herein. Where dCas9 proteins plus gRNAs are used, they may be delivered into cells by delivery subsystem 106, as directed by computing subsystem 105. Methods include one gRNA sequence, with one gene target, at a time; multiple gRNA sequences targeting multiple genes at a time; or as sequences of such deliveries over time.

In some embodiments, methods of expression level control within the present invention include the use of purposely mis-targeted gRNA to inhibit transcription of a target gene. In some embodiments, methods include the use of purposely altered gRNA to slightly mismatch target DNA sequence and therefore reduce mean residence time of the dCas9 RNP at the target site, and therefore modulate down expression. In some embodiments, methods include the use of different gRNA sequences to probe activation or inhibition of a particular gene. Doing this to groups of genes at the same time, for instance X genes*Y combinations, a single gRNA per gene in each well/sub-experiment). In some embodiments, methods use single-gene expression level modulation (vs just “on” or “off”) by different combinations of gRNAs. The methods include combinations, in different concentrations, of an on-target activating gRNA and an off-target inhibiting gRNA, with the relative concentrations setting the expression level of that gene. The methods include dilution of on-target activating gRNAs with “dummy” gRNAs that serve to lock up the scarce resource, the dCas9 proteins in the cell, and thereby modulate expression level. In some embodiments, methods use gene modulation timing control—at timescales shorter than the lifetime of dCas9+gRNA activity following delivery by a second delivery of gRNA that counters the effect of a first gRNA by targeting a different DNA site for a target gene.

In one embodiment, a gRNA designed to target the gene body is used to inhibit its transcription even with a dCas9-activator (CRISPRa) complex. Since CRISPRa is normally targeted to promoter ‘on-target’ regions to induce expression of a gene, when a dCas9 complex is targeted to nonpromoter intragenic ‘non-target’ regions, it can sterically hinder the binding of other complexes that mediate transcription. In this way, non-promoter intragenic gRNAs are used to decrease or even silence gene expression from a target locus.

In another embodiment, gRNA(s) targeting non-promoter intragenic regions is used simultaneously with gRNA(s) targeting the promoter of the same gene in order to modulate its expression when CRISPRa from the same Cas9 orthologue is present. The relative concentration of each gRNA proportional to the total number of gRNAs present within the cell determines the degree to which a single target gene is decreased in its ability to be expressed.

In another embodiment, gRNAs delivered at different timescales shorter than the lifetime of a CRISPR ribonucleoprotein (RNP) complex results in modulation of gene expression. For example, the activity of a first gRNA against an ‘on-targe’ site is overridden or reduced by a subsequent gRNA with a relatively higher concentration against an ‘off-target’ site as the concentration of the first gRNA is waning but before complete RNP turnover, resulting in consumption of remaining RNPs and rapid shut-off of activation from the first target locus.

In another embodiment, CRISPRa/i complexes from different Cas9 orthologues is used to discriminate whether gRNAs from one source species are activating vs. inhibitory in another species. For example, dCas9-VPR (CRISPRa) activates targets with Cas9-based gRNAs while dCpf1-KRAB (CRISPRi) represses targets with Cpf1-based gRNAs.

In one embodiment, gRNAs with different length protospacer sequences is used to modulate the activity of the associated CRISPR complex. gRNAs with 20-nt spacer sequences retain normal activity, while gRNAs with 19- to 14-nt spacers have reduced to no activity at the target locus (Kiani et al., Nat Meth, 2015). The relative concentration of 19- to 14-nt ‘dummy’ gRNAs to ‘on-target,’ ‘off-target,’ or non-functional regions is used to overwhelm or ‘lock-up’ CRISPR complexes from functionalizing 20-nt gRNAs to reduce gene expression at the 20-nt targets.

In one embodiment, 1-10 gRNAs targeting the promoter of a single gene are screened individually in separate wells to identify gRNAs with maximal CRISPRa-based activation of expression from the target locus. The top 1-10 gRNAs used for experimental screening are selected based on their computationally predicted ability to effectively activate a gene based on algorithms that incorporate chromatin, position, and sequence features, such as described in Konermann et al., 2015, Nature and Horlbeck et al., 2016, Elife, the contents of each of which are incorporated herein in their entirety.

In another embodiment, subsets and permutations of 1-5 gRNAs against the same target are experimentally screened in the same well to identify combinations of gRNAs that cumulatively have maximal effect on CRISPRa gene activation.

In another embodiment, 1-10 gRNAs per target against 1-5 targets are screened simultaneously in the same well for maximum CRISPRa activation of each of the 1-5 targets.

The computing subsystem 105 may therefore potentially determine (and direct, through selection of appropriate guides from library 103, and delivery by delivery subsystem 106) the start timing, level, and duration of gene modulations to be performed on each cell sample, in order to map out potential cell state routings.

### Delivery Verification and Barcodes

In certain embodiments, the invention provides three constructions. In an embodiment, constructs are delivered side-by-side with the gene modulators that mimics the diffusion of the gene modulators through the cellular membrane pores, thereby allowing reliable measurement of whether gene modulators were successfully delivered into the cell. In an embodiment, constructs are attached to the gene modulators being delivered into the cell, and subsequently separated from the gene modulators to perform their signaling/barcoding function. In an embodiment, constructs that are integral to the gene modulators are delivered into the cell.

Embodiments of the invention further comprise three modes.

One mode includes immediate confirmation that gene modulators were delivered into a cell. In an example, a fluorescent compound is delivered in the same solution as the gene modulators, and with similar diffusion properties. In another example, gene modulators are delivered with attached fluorescent components. Another example is a two-step version where a fluorescent reporter is cleaved from the gene modulator immediately following delivery into the cell, and can then be used to distinguish which cells received gene modulators.

Another mode includes confirmation of function of gene modulators (or parallel). An example includes constructs where gene modulator has a fluorescent reporter which is active when the gene modulator is active on the cellular DNA. In an example, a gene modulator that modulates another gene in the cell that functions as a “reporter,” for example a gene modulator that turns on an integrated green fluorescent protein gene.

Another mode includes recording for later analysis. “Barcoding” constructs that cause distinct sequences to be recorded in cellular DNA according to which gene modulators have been delivered, and in what sequence. Such sequences may be read out on a per-cell basis at the time of analysis and matched with cell expression profiles or functional profiles.

With immediate confirmation systems, delivery of gene modulators may be confirmed (with high certainty) almost immediately after delivery. For cells where delivery cannot be confirmed, the delivery system 106 may re-deliver gene modulators, potentially with higher energy levels/larger number of pulse repetitions (whichever parameter is applicable to the physical delivery modality being used), or it may kill and/or remove these cells to remove them from the experimental population.

With confirmation by gene expression, re-delivery, or alternative deliver programs as specified by computing subsystem 105, may be used on cells that do not confirm delivery and function. Alternatively, all cells that do not confirm delivery and function may be killed and/or removed from the sample, potentially with the same energy source as is used in the physical delivery system.

With “barcode” recording, DNA barcodes are inserted only when there is successful delivery of gene modulators, and barcodes correspond to these modulators, and potentially to the sequence or timing of delivery of these modulators. The barcodes are read out at the time of cell analysis. For example, barcode DNA may be transcribed in vitro into RNA, prior to single-cell RNA sequencing to measure cell transcriptomes (gene expression). Furthermore, these transcribed DNA-to-RNA signals at the single-cell level can be ligated as an adaptor to each mRNA and directly used as an indexing barcode during library preparation of a single cell's transcriptome within a partition to generate single-cell libraries with unique indices that are multiplexable for next generation sequencing.

Characteristics of delivery systems according to the invention include physical temporary membrane disruption (highly-specific delivery timing, delivery of bare cargo equals maximal timing specificity) and continuous culture format (minimum disruption equals minimum experiment cycle time).

Delivery systems within the invention include absorber-mediated pulsed laser, such as distributed absorbers and surface absorbers. The invention provides for spatially-addressed electroporation in gap, forward transfer of liquid towards cells, patterned substrate squeezing, and patterned gap-closing high-velocity liquid displacement.

In an embodiment, the invention provides bespoke spatial delivery of physical energy to cells through application of external signal. The invention images cell population to determine density, morphology, phenotype, and prior delivery success. Desired spatial energy density (cavitation, thermal, electric field) distribution corresponding to cell population distribution is determined. Energy is applied across the physical transducer according to this desired distribution, modulating by level of energy delivered per pulse in a pulsed and scanned system, or modulating the number of pulses per unit area in a pulsed & scanned system, or modulating by “pixel” in a system where energy is projected across the entire field.

For example, laser cavitation uses variable attenuator and multiple scans over surface with different pulse energies, with selective pulsing during an XY scan, to deliver different levels of energy over the cell culture. The invention uses such a system, but with variable pulse spacing in XY to deliver variable number of pulses per cell. The invention uses a high-speed attenuator/modulator, such as an acousto-optic modulator, to control energy on a per-pulse basis, so variable energy can be delivered in a single XY scan over the cell culture. The invention combines fast modulation and variable XY spacing to control both pulse energy and pulse density over the cell culture.

In an embodiment, the invention provides bespoke patterning of physical energy transfer surface prior to application of energy. The invention images cell population to determine density, morphology, phenotype, and prior delivery success. Desired spatial energy density (cavitation, thermal, electric field, mechanical pressure, fluid shear, acoustic pressure, puncture density) distribution corresponding to cell population is determined. The invention provides a pattern physical transducer accordingly. The physical transducer is positioned over the cells in culture, and external energy is applied.

Any suitable physical transducer may be used. For example, with cavitation, the invention selectively laser-ablates absorbing surface to achieve desired absorber/bubble nucleation pattern. For example, electroporation selectively laser-ablates conductive surface to achieve desired field intensity. For example, thermal methods selectively laser-ablate absorbing/heat transducing surface to achieve desired heating pattern. For example, squeezing selectively 3D prints/UV-cures layer(s) of material such as PDMS to apply appropriate mechanical pressure levels when lowered against cell-bearing surface. For example, liquid shear selectively 3D prints/UV-cures layer(s) of material such as PDMS to create appropriate high-velocity liquid flow field when lowered against cell-bearing surface. For example, sonoporation 3D prints bespoke acoustic metalens to direct incoming acoustic waves in a specific pattern onto the cell surface.

In an embodiment, the invention provides a spatial delivery mechanism.

Cargo can have multiple differentiation paths, such as by cell population characteristics. Delivery can be by cell density (mechanical properties), such as adjusted by cell size/morphology (e.g., area, or single-cell), adjust energy (laser, voltage, velocity), or adjust number of sites hit per cell (e.g., laser spots).

The invention may kill off cells to make space, due to phenotype, or for lack of delivery.

The invention may ablate the matrix to control area of attachment for cell patterning control, control direction/shape of growth, or redeposit additional matrix in later steps, if desired.

The invention may lift off cells (temp-sensitive matrix). An example includes ablating the outline to “cut out” and then work on the matrix. Special temp-sensitive layers may be used under the matrix, like Laminin. An example application includes retrieval of specific cells/colonies.

An application includes retrieval of sheets for formation of organoids, including layered 2D organoids; or folded, rolled structures (complex shape thinning/cutting is a big plus).

In an embodiment, the invention provides a reporter/barcode. An example of the reporter may include a fluorescent reporter to provide immediate proof of delivery, and may include use of molecule that mimics membrane crossing of active molecule or the use of fluorescent gRNA. An example of the reporter may include a fluorescent CRISPR action—action proof reporter, where a version of CRISPRa/CRISPRi/etc. fluoresces when on target DNA. An example of the reporter may include a fluorescent gene activation—parallel action proof reporter, where the reporter incorporates GFP gene, activates with gRNA delivered in parallel.

An example of the barcode, encoded in DNA—analysis may include parallel delivery, packaged delivery, and analysis. For parallel delivery, the construct is delivered in parallel with active gRNA that mimics membrane crossing on that gRNA, and causes a unique barcode to be encoded in cellular DNA for readout at analysis. For packaged delivery, the barcode that is attached to gRNA is cleaved after delivery to cell, to be recorded in DNA. For example, aCy5-attached barcode can be cleaved from gRNA in the cell and then recorded. Analysis may include parallel sequencing of barcode DNA with RNA transcriptome. Analysis may preferably include transcription of barcode DNA into RNA prior to RNA transcriptome sequencing. An example includes the use of T7 polymerase to do in-vitro transcription.

### iPSC Embodiment

An embodiment of the invention comprises cells in an initial state such as induced pluripotent stem cells (iPSCs). The cells have been genetically modified to express a dCas9 CRISPR activator (CRISPRa), and also green fluorescent protein (GFP), where the GFP gene can be activated or overexpressed using a CRISPRa with a specific gRNA.

The cells are plated onto 384 well plates that are fitted with an optically clear substrate that has regularly spaced optical absorption/bubble nucleation sites on the well-facing surface, for example patches of metallic absorber with a sufficiently rough top surface to promote microsecond bubble nucleation upon exposure to a short laser pulse. The surface is pre-coated with a matrix to promote healthy cell growth (for example Matrigel™ or laminin).

A computing subsystem uses a model of previous differentiation experiment outcomes to compute the most likely programs (gene modulation sequences) to differentiate iPSCs towards the target functional cell type, selecting the top 96 potential programs. Each program will be applied to 4 wells on the plate, where 3 wells serve to provide a time sequence (2 intermediate time points, and a final timepoint), and 1 well is a spare in the case that one well is found to be defective in the course of the experiment. Prior to each gene modulator delivery step, the well plate is imaged to assess cell count, density, and morphology in a spatially-specific manner.

For each well, liquid containing the gene modulator(s) to be inserted during the step is prepared. This liquid may contain one or more gRNAs targeting genes to be activated or repressed, in the manner described above. A “tracer” may also be added to the liquid, where the tracer compound has similar trans-membrane diffusion characteristics as the gRNA constructs, and is measurable immediately post-delivery via microscopy or fluorescence microscopy. Alternatively, this may be accomplished by a tracer that is attached to gRNAs. A gRNA targeting an integrated GFP gene may be added, either temporarily attached to active gRNAs or separately. The GFP signal serves to confirm delivery of active gRNAs within hours of the delivery step. A construct for DNA barcoding that is unique to the step being performed may be added. The barcode is recorded in cellular DNA is read out at terminal single-cell analysis.

Delivery of gene modulators (individual gRNAs, or combinations of gRNAs) is done by removing cell medium from a well, placing a solution with the gRNAs into the well, and scanning the well with a pulsed laser, where the laser energy and pulse density may be determined by local (or per-cell) cell density, morphology, etc. The laser pulses cause microbubbles that stress cell membranes and make them temporarily permeable to gRNAs. The gRNAs and other compounds described above diffuse into the cells that were successfully permeated. The cell membranes re-seal within seconds to minutes. Where cell density is deemed to be too high, or where cells look unhealthy/not appropriate for further experimentation, the cells may be destroyed and removed using higher laser power/pulse repetitions.

Immediately following the delivery step (including diffusion time), cell media is re-added to the well, potentially with multiple changes to remove any gRNA or other compounds. Where a “tracer” was used, imaging is used following the delivery to measure delivery to cells. Where delivery is incomplete (cells with no tracer delivery are detected), another laser delivery step may be used to re-deliver to these cells (potentially with higher energies and/or pulse repetitions), or to destroy these cells and remove them from the sample.

In the hours following delivery, where a gRNA targeting activation of an optionally integrated GFP gene has been delivered together with “programming” gRNAs, expression of GFP at a per-cell level may be measured by microscopy; those cells that do not express the GFP—and therefore did not successfully have functional gRNA delivered (or are not properly expression dCas9)—may be removed from the sample using the laser scanning system, with high pulse energies and/or repetitions. This further ensures that only cells with intact programming sequences proliferate and differentiate. Such measurement and removal may be done in subsequent delivery steps.

One or more wells within a 4-well set may be sent to analysis at intermediate timepoints, where cell expression may be measured by a range of techniques; the results may be used post-experiment to complete “routing maps” in the differentiation model in the computing system. The results may be used in “real time” (within the experimental timeline of the well plate) to discard experiments that do not show transition of cell states in the preferred direction. The results may be used in “real time” by the computing subsystem to recompute optimal programs for differentiation from the intermediate state, e.g. altering the gRNAs and/or timepoints used for a particular sample in the remainder of the experiment. Multiple modes of analysis may be used per well. Fast analysis techniques (for example, bulk qPCR) may be used on a portion of the cells to assess gene expression/state for “real time” guidance. More time-consuming techniques such as single-cell RNA sequencing may be used on other cells to build a detailed map of cell differentiation, to optimize future experiments.

In some experiments, delivery steps may be done with spatial patterning in order to purposely create a diversity of cell state transitions, even within a single well. The different programs applied may be recorded through barcoding as described above. This may be done to create cell populations with multiple cell types, or simply to increase experimental throughput in the system.

Data from the analysis step is added to the computing system database to build relationship/state map models used to further refine the gene modulation programs, until a desired cell state (from an in vitro analysis standpoint) is achieved. Functional testing may be subsequently performed, and degree of function incorporated into the computing model, for example to relate gene expression levels to functional indicators and further refine the target cell profile within the present system. Additionally, downstream data from in vivo experimentation may be incorporated into such a model as well, again to refine the target of the system as it optimizes the differentiation program.

### Directed Cell Fate Specification

Methods and systems of the invention may be used for directed cell fate specification.

In an embodiment, the invention provides methods and systems for directing cell fate. Methods of the invention include identifying genes that are involved in directing cell fate specification of a desired cell type. Using methods of the invention, a minimal number genes determined to be responsible for directing cell differentiation of the desired cell type are selected as target genes and sequences of a minimum number of guide RNAs corresponding to each of the genes are identified. Methods of the invention include introducing into stem cells, such as pluripotent stem cells, complexes that include the guide RNAs and a Cas protein to cause differentiation into the desired cell type. The differentiated cells are characterized by methods to identify cell traits and their cell types identified by comparing known cell traits of cell types to that of the differentiated cells. If the desired cell type is not achieved in the first cycle of design-test-characterize, the cycle is repeated and each time a minimal number of the genes is identified as being responsible for directing cell differentiation of the desired cell type. The genes identified in the first cycle, may also be identified in subsequent cycles. Cycles of the method may be repeated to further enhance a phenotype of the cell type.

One approach to identify genes suspected to direct cell fate of a desired cell type is to perform a literature search. In another approach, inputs from various data sources are analyzed using bioinformatics analysis and genes are identified as directing cell fate of the desired cell type. A minimal number of the genes are selected and a guide RNA sequences to target each of the genes are then identified by analyzing the data. In one approach, identifying the minimum guide RNAs includes introducing into each of a plurality of cells a Cas protein and a guide RNA complex to produce a viable cell, or progeny thereof, measuring gene expression of the target in the viable cell to identify a minimum number of guide RNAs causing optimal gene expression of the target gene. In another approach the minimum set of guide RNAs are identified by bioinformatics analysis of the data. In another approach, sequences of the gRNAs may already be known, for example, if the gRNAs were designed from a database or purchased for use in a screening method.

Once the minimum set of guide RNAs are identified, they are complexed with a Cas protein and introduced to the stem cells to direct cell differentiation of the desired cell type.

A Cas9 that is catalytically active cleaves DNA via its HNH and RuvC nuclease domains. When the Cas9 nuclease has two functional domains and both of these domains are active, the Cas9 causes a double stranded break in the DNA. Thus, a Cas protein may be targeted to a specific location by forming a complex with a gRNA that includes a ˜20-bp guide sequence that is substantially complementary to a genetic locus. It is understood that gRNA includes gRNA with a trans-activating RNA (tracrRNA) as well as the use of a single guide RNA (sgRNA). In contrast, in dCas9, the HNH and RuvC nuclease domains are modified to disable their DNA cleaving activity, resulting in a dCas that retains its DNA binding ability but not its DNA cleaving activity. For example, point mutations may be introduced at catalytic residues (D10A and H840A) of the gene encoding Cas9. Complexes including dCas9, gRNA, and one or more effector domains can therefore take advantage of the DNA binding activity of the Cas9 protein and the DNA targeting ability of gRNA to intentionally bring the effector domains to target loci to cause cell differentiation into the target phenotype.

It is appreciated that any Cas protein that forms a complex with and is guided by the gRNA may be used, for example, Class II Cas proteins such as Cas9 and Cpf1. Cas proteins with single-subunit effectors are known as Class 2. These are then subdivided even further into type II (e.g., Cas9) and type V (e.g., Cpf1). Cas proteins include Cas9, Cpf1, C2c1, C2c3, and C2c2, and modified versions of Cas9, Cpf1, C2c1, C2c3, and C2c2, such as a nuclease with an amino acid sequence that is different, but at least about 85% similar to, an amino acid sequence of wild-type Cas9, Cpf1, C2c1, C2c3, or C2c2, or a Cas9, Cpf1, C2c1, C2c3, or C2c2 protein with a linked to an accessory element such as another polypeptide or protein domain (e.g., within a recombinant fusion protein or linked via an amino acid side-chain) or other molecule or agent.

C2c1 (Class 2, candidate 1) is a type V-B Cas endonuclease that has been found. Examples of C2c1 have been indicated to be functional in E. coli. tracrRNAs (short RNAs that help separate the CRISPR array into individual spacers, or crRNAs) were required. As is the case for Cas9, with C2c1, the tracrRNA may be fused to the crRNA to make a single short guide, or sgRNA. C2c1 targets DNA with a 5′ PAM sequence TTN.

C2c3 (Class 2, candidate 3) is a type V-C Cas endonuclease that clusters with C2c1 and Cpf1 within type V. C2c2 was found in metagenomic sequences, and the species is not known.

C2c2 (Class 2, candidate 2) is a type VI Cas endonuclease. C2c2 has been indicated to make mature crRNAs in E. coli. See Shmakov, 2015, Discovery and functional characterization of diverse class 2 CRISPR-Cas systems, Mol Cell 60(3):385-397, incorporated by reference.

In one embodiment, the complexes introduced include a dCas9 protein that forms a complex with the gRNAs and effector domains. For example, the effector domain may be an activator, an inhibitor, or a domain that recruits coactivator or corepressor proteins to the complex, for instance, by acting as a scaffold.

Examples of effector domains that acts as activators include the VP16 activation domain (VP16), VP48 (three copies of VP16), VP64 (four copies of VP16), VP96 (six copies of VP16), VP160 (ten copies of VP16), VP192 (twelve copies of VP16), the p65 activation domain (p65AD), VPH (VP192, p65, and heat shock factor 1 (HSF1)), VPPH (VP192, a catalytic core of human acetyltransferase p300 (p300), p65, and HSF1), and VPR64. VPR64 is a tripartite activator domain that includes VP64, p65AD, and the Epstein-Barr virus R transactivator (Rta). Examples of effector domains that acts as inhibitors include the Krüppel-associated box (KRAB), four concatenated mSin3 interaction domains collectively labelled (SID4X), and max-interacting protein 1 (MXI1).

An example of an effector domain that recruits subsequent effector domains is a SunTag. In a dCas9-SunTag complex, dCas9 may be fused with a SunTag array made of ten copies of a small peptide epitope. The SunTag array acts as a scaffold to recruit multiple copies of effector proteins. The effector proteins recruited may be, for example, VP64 activator proteins fused to a cognate single-chain variable fragment (scFV).

In another example, a synergistic activation mediator (SAM) effector domain is included in the complexes, in which two bacteriophage MS2 RNA aptamers (MS2s) are added to the tetraloop and second stem-loop of the gRNA complexed with dCas9. These MS2 RNA aptamers are able to recruit MS2 coat proteins (MCPs). MCPs are MS2 coat proteins fused to VP64, p65AD or HSF1 activators.

In various embodiments, selection marker domains may be included to assist in selecting and enriching for cells with stable uptake of the complexes. For example, the selection marker may be a fluorescent marker (GFP), or drug resistant marker (puromycin). If such selection markers are used, cells may be selected for stable uptake of the complexes, for example, by fluorescence-activated cell sorting (FACS) if a GFP selection marker was employed or by drug screening if the puromycin selection marker was employed.

In some embodiments, effector domains may be directly fused to dCas forming, for example, dCas9-VP16, dCas9-VP48, dCas9-VP64, dCas9-VP96, dCas9-VP160, dCas9-p65, dCas9-VPH, dCas9-VPPH, dCas9-VPR64, dCas9-KRAB, dCas9-SID4X, or dCas9-MXI1. In other embodiments, such as dCas9-SunTag and dCas9-SAM, the effector domains are not directly fused to dCas9, but instead recruit other proteins to cause an activating or inhibiting activity. It is understood that include effector domains may manipulate epigenetic modifications such as histone acetylation or methylation and DNA methylation. For example, inhibiting activity may be caused by dCas9-LSD1 (Lys-specific histone demethylase 1) and activating activity may be caused by dCas9-p300.

The complexes may be introduced into the stem cells in various ways and by any suitable method, for example, by transfection or transduction. The complexes may be introduced into the cells as an active protein—or ribonucleoprotein (RNP) in the case of a Cas-type nuclease—or encoded in a vector, such as a plasmid or mRNA, in a viral vector, such as adeno-associated virus (AAV), or in a lipid or solid nanoparticle. The complexes may be transfected into cells by various methods, including viral vectors and non-viral vectors. Viral vectors may include retroviruses, lentiviruses, adenoviruses, and adeno-associated viruses. It should be appreciated that any viral vector may be incorporated into the present invention to effectuate delivery of the complex into a cell. Use of viral vectors as delivery vectors are known in the art. See for example U.S. Pub. 2009/0017543, incorporated by reference. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355, each incorporated by reference) and lipofection reagents are sold commercially (e.g., Transfectam and Lipofectin).

In one embodiment, dCas, effector domains, and gRNAs are transcribed in vitro, complexed to form an RNP complex, and introduced into the stem cells by any suitable method, for instance, electroporation or cationic lipid transfection. dCas and effector domains may also be transcribed in vitro and introduced into the stem cells separate from transcribed gRNAs. In another embodiment, mRNA encoding dCas may be co-introduced with the gRNAs. The dCas encoded by the mRNA may include one or more effector domains. Also, the dCas may be constitutively expressed. The mRNA encoding dCas may also encode gRNA. In another embodiment, dCas and gRNAs are introduced into stem cells by transduction with or separate from the gRNAs. For example, dCas and gRNA may be attached to a single lentiviral backbone and introduced by lentiviral transduction.

Methods of the disclosure identify guide RNAs that differentiate stem cells to a target cell type. Methods may include predicting the sequence of guide RNAs that target promotor areas of genes identified as involved in differentiating cells to a specified differentiated cell type, and then using the methods to identify the minimum effective set (e.g., 1 to 5 per gene for 1 to about 5 different genes associated with the specified differentiated cell type) of guide RNAs, in which the effective set can be delivered with a CRISPRa/i protein to effectively differentiate stem cells into the specified differentiated cell type. Selecting the guide RNAs can include a process that includes (1) first a literature search to identify genes suspected to be involved in differentiating cells to the specified differentiated cell type followed by (2) a genomic database search (e.g., in GenBank or Ensembl) to identify suitable guide RNA targets (e.g., unique or nearly-unique 20 base stretches, adjacent to a protospacer adjacent motif, within putative promoter regions of genes identified in step 1). Searching the databases may be performed by computer software such as a Perl script or Python code that applies the rules for Cas endonuclease guide RNA targeting and identification of promoter regions for coding strands to identify putative targets. That same code may perform step 1 (so-called literature search) by searching keywords in GenBank annotations to identify coding regions that have been labelled with keywords specific to a desired phenotype or cell type. The set of guide RNAs identified by the in silico selection methodology (steps 1 and/or 2) may then be provided as RNA molecules for delivery to the stem cells. The desired RNAs may be ordered e.g., from a service such as Integrated DNA Technologies, Inc. (Skokie, Ill.) or synthesizing the RNAs on a synthesis instrument. The guide RNAs are introduced into the stem cells with the dCas protein linked to a transcription regulator (e.g., an effector domain). The process may also include (3) analysis of the data to identify a temporal sequence of expression to direct cell fate specification of a subtype of the desired cell type. In yet another embodiment, the analysis for step 3 may also include implementation of software/algorithms to reconstruct and verify the subtype is achieved. If the subtype is not achieved, steps 1-3 are repeated until the subtype is achieved.

The dCas-linked effector domain protein and the guide RNA may be delivered into stem cells in any suitable format and using any suitable delivery technology. For example, the protein may be introduced as a DNA vector (e.g., plasmid or viral vector), in the mRNA sense, or as a formed protein. The guide RNA may be introduced in the same or different DNA vector, as a free guide RNA, or complexed with the protein in the form of RNP. Whichever format is used, the molecular structures to be delivered may further be complexed with, linked to, or encapsulated by any suitable delivery reagent such as one or more nanoparticles (such as a solid lipid nanoparticle, a micelle, metal particles, polymer particles, or a liposome), PEG, or biological macromolecules such as sugars or intra-cellular trafficking proteins such as nuclear localization signals. The molecule structures to be delivered may further be delivered using any suitable technology. In preferred embodiments, screening methods of the disclosure scale up to high throughput, and allow multiple replicates to be performed in parallel (e.g., tens or dozens or greater of 384-well plates are filled with experimental aliquots using, e.g., liquid handling robots). Delivery of the molecular structures to such quantities of stem cells may be best served using a technology that scales up well to high-throughput applications such as laser excitation of plasmogenic substrates. In such technologies, a reactive substrate is presented in proximity to stem cells and the payload to be delivered (e.g., dCas-effector domain RNP, or a nucleic acid encoding the dCas-effector domain RNP, and guide RNAs). The substrate is excited with a laser. Preferably, the substrate includes physical structures such as tetrahedral peaks (e.g., a grid comprising thousands of peaks over an area of plasmogenic material on the order of 10 mm×10 mm. Laser excitation of the surface induces temporary poration of the stem cells, allowing the payload to diffuse into the stem cells. Such a technology can provide the throughput necessary to introduce a dCas protein linked to a transcription regulator, or nucleic acid encoding the same, and a guide RNA, into each of thousands or tens of thousands or more stem cells, allowing for similar quantities of guide RNAs to be synthesized (e.g., using a benchtop RNA oligo synthesis instrument) and delivered to the stem cells. This technology for efficiently delivering functional cargo to millions of cells within minutes may be offered under the trademark NANOLAZE. See Saklayaen, 2017, Intracellular delivery using nanosecond-laser excitation of large-area plasmonic substrates, ACS Nano 11:3671-3680, incorporated by reference.

Once the payload is delivered into the stem cells, methods of the disclosure include differentiating one or more of those stem cells into cells of a desired cell type. In other embodiments, the stem cells are further differentiated into cells of a desired phenotype.

Next, differentiated cells having the desired cell type may be identified. Cell types are identified by specific cell traits that have been previously identified as characteristic of a certain cell type. Cell traits may include cell morphology, chromosome analysis, DNA analysis, protein expression, RNA expression, enzyme activity, cell-surface markers, or a combination thereof. Each of the differentiated cells produced by methods of the invention may be characterized by cell traits. Characterizing the cells may include identifying cell traits by staining the cells with a marker for the desired characteristic, and sorting the cells using, for example, a fluorescence-activated cell sorting instrument, a magnetic bead-based purification, others, or a combination thereof. In another embodiment, characterizing the cells may include identifying cell traits by measuring gene expression in the cell or progeny thereof. Gene expression includes one or more of: quantifying expression levels via RNA-Sequencing; measuring gene expression via single-cell RNA sequencing; or evaluating DNA-protein interaction via chromatin immunoprecipitation and DNA sequencing (ChIP-seq). The methods may include determining fold-change in expression level of a transcript associated with a marker by normalizing read counts from the measuring against control read counts. The methods may also include comparing transcriptomes of individual cells to assess transcriptional similarities and differences between the cells.

The cell type of each of the differentiated cells may be determined by comparing the identified traits of each of the cells to the known traits of a cell type. The methods may also include identifying cell type by comparing transcriptomes of the cells to assess transcriptional similarities and differences between the cells and may include clustering like cells. In an embodiment, the desired trait includes a specified differentiated cell type and the marker includes a protein expressed by the differentiated cell type. In an example, the desired trait may be a neuronal phenotype, and marker one or more of the presence of beta III tubulin and DAPI and the absence of Oct4. In another example, the desired trait may include an inducible neuron phenotype, and the marker the presence of beta III tubulin.

Optionally, the cell type of the differentiated cells can be identified by other methods such as FACS or drug screening, depending on the selection markers used. In various embodiments, the cells may be sorted as whole populations for analysis or as single cells for isogenic expansion or single-cell analysis. For example, in single-cell analysis, a single cell with the target phenotype is lysed, its genomic DNA is isolated, whole-genome-amplification is performed, a sequencing library is constructed, and the DNA of that single cell is sequenced. In certain embodiments, sequencing is by single-cell NGS. Single-cell NGS generally refers to non-Sanger-based high throughput DNA sequencing technologies applied to the genome of a single cell, in which many (i.e., thousands, millions, or billions) of DNA strands can be sequenced in parallel. Examples of such NGS sequencing includes platforms produced by Illumina (e.g., HiSeq, MiSeq, NextSeq, MiniSeq, and iSeq 100), Pacific Biosciences (e.g., Sequel and RSII), and Ion Torrent by ThermoFisher (e.g., Ion S5, Ion Proton, Ion PGM, and Ion Chef systems). It is understood that any suitable next-generation DNA sequencing platform may be used for single-cell NGS as described herein. Machine learning may be applied to the data obtained from the characterization steps of any of the methodologies used to characterize the cell identity to predict combinations of genes to be targeted by the complexes. For example, machine learning may be used to predict networks of interrelated genes whose alteration activates, represses, or modifies transcriptional networks to produce the desired cell types. When machine learning is applied, training data for the machine learning may include data obtained from systems of algorithms, publications, public data sets (e.g., gene expression data sets), cell type profiles, scRNA-seq (single-cell RNA sequencing) expression data, results of internal analysis, and any other data relevant data sources.

One way of making use of the disclosed methods of the invention may be to optionally utilize the output of a trajectory inference system of algorithms, CellRouter (Lummertz da Rocha, 2018) or that of DPT (diffusion pseudotime) in Nature Methods, or that of Monocle, published in Nature Biotechnology in 2014 and Nature Methods 2018 in August 2017 to identify additional target genes to differentiate stem cells to the desired cell type. The outputs can be added to the data for more refined analysis.

In an example, a cluster analysis of the invention clusters single-cells such that each cluster shows differential gene expression signatures. Genes preferentially expressed in each cluster, including known neuronal genes, have shared/similar features such as gene expression, phenotype, and genetic pathways. From the cluster analysis, one can identify networks of genes that exhibit features with a high degree of similarity (relatedness). Based on the high degree of similarity, cell type lineage trajectories and the associated genes can be identified. By way of example, graph theory algorithms can be utilized, and one way of making use of the methods of the invention is to utilize the outputs of those described in CellRouter, to identify cell network similarities. Cell types of differentiated cells are identified by identifying transcriptome similarities amongst the cells, where the cell clusters are representative of different cell types of the lineage. Community-detection algorithms (e.g., the Louvain method) may be used to identify inter-connected cells, and therefore define cell types. As such, methods of the invention utilize graph theory algorithms to cluster cell types. Clusters of the cell types can be depicted visually in the graph, such as t-SNE plot. Using previously identified cell type gene signatures, the cell types can be further categorized, for example.

Another way of making use of the disclosed methods of the invention is to utilize flow network algorithms, such as those described by CellRouter to then identify cell type trajectories. The structure of the network is a directed graph and the vertices are called nodes and the edges are called arcs and represent connections between the nodes. G=(V, E), where V is a set of vertices and E is a set of V's edges—a subset of V×V—together with a non-negative function c: V×V [symbol] ∞, is the capacity function. If two nodes in G are distinguished, a source s and a sink t, then (G, c, s, t) is called a flow network. A flow must satisfy the restriction that the amount of flow into a node equals the amount of flow out of it, unless it is a source, which has only outgoing flow, or sink, which has only incoming flow. Transformations known in the art can be used to optimize the network. Here, each node represents a single-cell and each edge connects cells that are phenotypically similar. Phenotypic similarities are quantified by the edge's weights. As such, the entire network, or graph will provide cell-to-cell similarities, thereby identifying paths connecting cell types (the cell clusters) and therefore defining differentiation trajectories.

Using a gene expression analysis of the data sets of the cell clusters, significant overlaps, or commonalities, in the data, for example, overlaps in genetic content are identified. Gene expression amongst different cell types, or cell clusters, can be compared to identify overlap of genes amongst the cell types. In certain aspects of the invention, the gene modules identify an overall functional congruity between cell clusters allowing for the identification gene expression patterns. In a preferred embodiment, differentiation pathways between the cell types are identified. The genes are typically mammalian genes. The mammalian genes may correspond to mouse genes, human genes, or a combination thereof. Feature data (such as gene expression, phenotype, gene pathway, etc.) and genes may be used to form a matrix that will be used to exhibit the trajectory inference analysis. For example, the feature data is pre-processed to express each domain as a row and each feature as a column (or vice versa). For domains with continuous values such as gene expression, the features are the individual cells of which gene expression was measured, and each value in the matrix (Xij) represents the expression of gene i in a cell j. For domains with categorical values such as phenotypes, the features are the individual phenotypes, and each value in the matrix (Xij) is a binary indicator representing whether gene i is associated with phenotype j. All of the domain specific matrices are then combined column-wise. A distance metric is then applied to each pair of rows and each pair of columns in the matrix. In certain embodiments, the distance metric is ‘Distance=1-correlation’. However, it is understood that other standard distance metrics could be used (e.g. Euclidean).

Generally speaking, after a graph-based clustering analysis is applied to the gene expression data, the gene “clusters” can be displayed against certain feature categories (e.g. phenotype/gene expression ‘category’), which are then clustered to reflect commonality. In other embodiments, the gene modules (or clusters) are displayed against the cell different cell type trajectories. For example, phenotypes of immature dopaminergic neurons (hDA0) are grouped together in one cluster, and phenotypes of mature dopaminergic neurons (hDA1 and hDA2) patterning, morphology and growth are grouped in a separate cluster, etc. The degree of relatedness or commonality between the clustered cells and the cluster-specific genes (as determined by the cluster analysis) can then be highlighted on the resulting cluster matrix. For example, red may be used to indicate that the gene is associated with morphology and/or is expressed at high levels in the associated cell type indicated on the opposite axis; whereas blue may be used to indicate that the gene is associated with morphology, but and/or is expressed at low levels in a different cell type.

Methods of the invention assess several features (or parameters) of genes in order to determine their relationship to a cell type differentiation trajectory. The method includes ordering cell types from early to late stage differentiation. In certain embodiments, the features include gene expression, phenotypes, gene pathways, and a combination thereof. In a preferred embodiment, the trajectory is a developmental trajectory of a cell from an immature cell type to a mature cell type. As such, the cell types are ordered along a pseudo-timeline of cell development. In another embodiment, the trajectory is an intermediate trajectory from one cell type to another cell type. In another embodiment, the ranked genes are the genes necessary to direct cell differentiation. By clustering cells and identifying cluster-specific genes into feature specific groups and color-coding genes with high degree of relatedness, the resulting cluster matrix of the invention advantageously allows for visualization of groups of genes (the modules) that are strongly associated with phenotypes relating to particular cells (i.e. clusters of interest). Thus, cluster matrices of the invention allow one to quickly identify a detailed mapping of cellular differentiation pathways based on cell types' shown association (cluster) with one another. This mapping further allows for identification of genes responsible for establishing genetic regulatory networks (GRN) for a cell subtype, by further mapping the gene along the trajectories.

Methods of the invention include inputting the results of scoring the genes of the GRN into the systems described herein to predict transcriptional regulators. For example, a GRN score maybe assigned to each gene by implementing the CellRouter algorithm. The genes are assigned a GRN score based on their correlation with their progression of the identified trajectory, their correlation of their predicted gene targets, and the extent to which target genes are regulated during a particular trajectory. The up-regulated genes with the highest score and down-regulated genes with the highest score are selected and mapped to the genes of downstream GRNs to identify genes responsible for the cell fate. In an embodiment of the invention, at least 1 of the top ranked up-regulated genes and at least 1 of the down-regulated genes are identified. In a preferred embodiment, 10 to 20 up-regulated genes with the highest score and 10 to 20 down-regulated genes with the highest score are selected. The method also includes plotting the expression levels of the genes across the pseudo-timeline to identify inflection points in gene expression along the trajectory. As such, the method identifies genes and the temporal sequence of the gene expression to direct cell fate. In another embodiment, the genes identified are temporally expressed to direct the cell fate. The temporal expression of the genes regulates the expression of target genes associated with a specific cell type.

As a result of integrating both the GRN scoring and the pseudo-timeline (or temporal sequence) into the methods described herein, a minimum number of genes and a temporal sequence of expression of the genes to direct maturation of a cell are identified.

To determine if the identified genes are capable of differentiating the stem cell into the desired cell type, methods of the invention analyze data obtained from a plurality of sources, including, for example, the output of CellRouter, to identify sequences of a corresponding minimum number of guide RNAs and induce stem cells with the guide RNAs complexed to Cas protein and may be repeated until the subtype is verified by the method. In another embodiment, the methods of the invention may be performed in silico, and may be repeated until the subtype is verified by the method.

The data obtained from the methods are inputted into the database and machine learning may also be applied to the data produced by analysis performed on the system or data sets inputted into the system to identify limited targets and corresponding minimal number of gRNAs to produce a desired cell type. For example, machine learning may be used to predict networks of interrelated genes whose alteration activates, represses, or modifies transcriptional networks to produce the target phenotypes using the database. When machine learning is applied, training data for the machine learning may include data from gene expression analysis, as well as other publicly-available sequencing data from various stages of the natural development of the starting cells of the target phenotype. In an example, features of the target phenotype may then be split into individual parameters to categorize gRNAs identified or predicted to be involved in causing those phenotypic features in the stem cells.

In another example, machine learning may be used to identify specific temporal sequence of activating and/or inhibiting target genes to differentiate cells. For example, the temporal sequence may include the introduction of a first set of one or more guide RNAs during a first period comprising one or more hours or days followed by introduction a second set of one or more guide RNAs during a second period comprising one or more hours or days. Optionally, the first set of one or more guide RNAs and the second set of one or more guide RNAs comprise wholly different guide RNAs and/or the first period and the second period do or do not overlap in time. In some embodiments, CRISPRa/i is used against a first set of targets during the first period, the first period comprising at least two days, and using CRISPRa/i against a second set of targets during the second period to differentiate the one of the plurality of stem cells into a dopaminergic neuron. As such the invention includes methods of introducing RNAs (e.g., with the dCas protein linked to the regulator) into at least one of the plurality of stem cells in a temporal sequence.

Upon identifying genetic targets and the temporal sequence, the machine learning system provides a report with a program for using the targets and the temporal sequence to allow for specified cell fate engineering. The program is for the sequential delivery of CRISPRa/i RNPs or transcription factors to the starting cell type (e.g. human pluripotent stem cells), along with sequence of the identified targets encoded in vectors with conditional modules that recapitulate the program necessary to derive the desired cell type/subtype. The report can also identify a gene module to be used on any type of cell to effectuate a specific phenotype.

Vectors can include integratable viral (e.g. lentivirus) non-viral (e.g. PiggyBac) methods, or non-integratable viral (e.g. Sendai virus) and non-viral (e.g. episomal) methods. In one embodiment, hiPSCs receive an episomal vector containing transcriptional regulators under different inducible promoters where the relative timing of expression of each factor or set of factors is achieved through exposure of the cells to different inducers in varying combinations across time to achieve cell fate specification. In another embodiment, an episomal vector contains a constitutively expressed CRISPRa/i with a guide RNA array where components of the array are inducible in varying combinations across time to achieve cell fate specification of the subtype.

In certain embodiments the invention allows for mapping of cell differentiation pathways or trajectories, for example from one cell type to a specific phenotype or subtype of the cell type and the cell subtypes are ordered from early to late stage development by clustering the cells by subtype. The ordering of the cell subtypes identifies a pseudo-timeline of cell development for the cell type. Those cell differentiation pathways allow the system to identify genes (or transcriptional factors) responsible for establishing the GRN. The genes are assigned a GRN score according and the genes (both up-regulated and down-regulated) with the top scores are mapped to downstream GRNs. Such mapping identifies the minimum number of to effectuate cell differentiation of a particular cell subtype. By further plotting the expression levels of the minimum number of genes across the pseudo-timeline, the system identifies the temporal expression sequence of the genes. Methods and systems of the presently disclosed invention allow for cell fate engineering by identifying a minimum set of target genes and their minimal effectors capable of specifying cell fate between any given cell type.

### Examples

### 1. Identification of Factors and Effectors

Methods and systems of the invention are utilized to identify gene targets and guide RNAs to differentiate stem cells (e.g., iPSC) into neurons, and more specifically, into dopaminergic neurons in the following example. It is understood that the methods disclosed may generally be applied to any starting cell type to produce any target phenotype and that any combination of gRNAs and effector domains to cause CRISPRa activation activity or CRISPRi inhibition activity may be employed. Preferably the transcription regulator under guidance of the dCas protein and one or more guide RNAs will cause differentiation of one of the plurality of stem cells into the viable cell or progeny thereof such that correlating the change in gene expression with the targets of the guide RNAs identifies loci to target by CRISPRa and/or CRISPRi to differentiate pluripotent stem cells into a target cell type. As the starting cells for screening, a dCas9-VPR stable iPSC cell line is created. Alternatively, any existing iPS cell line may be used.

First, NEUROD1 and NEUROG3 were identified by methods of the invention to be drivers of neural differentiation. Specifically, these gene targets were identified by bioinformatics analysis of data from a plurality of sources.

Next, using the methods and systems of the invention, the sequences (Table 1) of four (4) sgRNAs for each target gene (NEUROD1 and NEUROG3) were identified and predicted to have maximum activation in a CRISPRa system using bioinformatics analysis. The sgRNAs were then designed using methods known in the art. These synthetic sgRNAs were then transfected, either pooled or individually, into an iPSC line stably expressing the CRISPRa complex dCas9-VPR. Optionally, the dCas9-VPR complex can be introduced into iPSCs using viral vectors (e.g., lentiviral) or transposable elements (e.g., piggyBac).

TABLE 1

| | | CRISPRa |
|---|---|---|
| Target Gene | sgRNA ID | Protospacer Sequence |
| NEUROD1 | NEUROD1_1 | AGCAAGGCGTGGGGAGAAGT |
| | NEUROD1_2 | GGGGAGCGGTTGTCGGAGGA |
| | NEUROD1_3 | GCGGGAGACGAGCAAGGCGT |
| | NEUROD1_4 | GTGAGGGGAGCGGTTGTCGG |
| NEUROG3 | NEUROG3_1 | CACAGCTGGATTCCGGACAA |
| | NEUROG3_2 | CCTCGAGAGAGCAAACAGAG |
| | NEUROG3_3 | GGACAAAGGGCCGGGGTCGG |
| | NEUROG3_4 | CCACACGAGGCTCTTCTCAC |

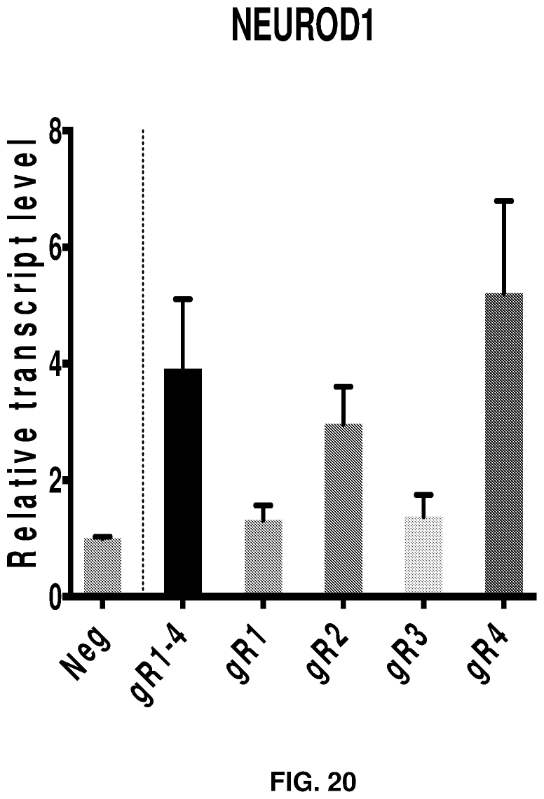

For this example, to confirm that the target genes were activated relative to a non-targeting control, qPCR gene expression analysis was performed. FIG. 20 illustrates the RT-qPCR data that were normalized to endogenous control gene ACTB. The relative transcript levels are compared to samples transfected with a non-targeting negative control sgRNA. Error bars represent standard error of the mean (SEM) from three biologic replicates with three technical replicates each. Such analysis identified NEUROD1 sgRNA #4 and NEUROG3 sgRNA #2 as the optimal guide RNAs of each gene, respectively. The sequences of the optimal guide RNAs identified by gene expression analysis are identified as such and inputted back to the machine learning system of the invention. Optionally, the gene expression analysis does not need to be performed and pools of 4-5 sgRNAs per gene target identified by the methods of the invention can be used for cell differentiation.

### 2. Testing Identified Effectors for Differentiation Ability

Next, the identified sgRNAs were delivered to stem cells to determine their ability to direct cell differentiation towards a target cell type. Specifically, the guide RNAs (NEUROD1_4 and NEUROG3_2) delivered into the stem cells by lentiviral, express antibiotic resistance to puromycin. Other delivery methods include transient transfection (e.g., lipofection, electroporation, or NanoLaze) or stable delivery by virus (e.g., lentiviral, sendai). The induced cells are then enriched for those successfully receiving sgRNAs by FACS or drug selection.

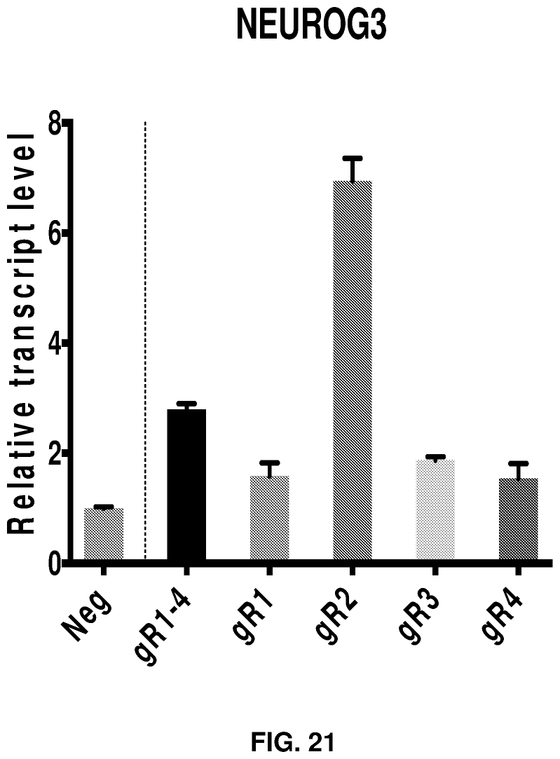

For the purpose of generating the desired neuronal cells, at day 0 dCas9-VPR iPSCs were plated at varying concentrations (3.5K, 7K, 10K, 15K) in a 96-well format and were transduced at a MOI of 10 with either each sgRNA alone, or in combination. Puromycin selection was applied at day 1 to select for cells successfully transduced with lentiviral sgRNAs. More mature cells were then collected at day 3 and at day 7 as depicted in the timeline in FIG. 21.

The stem cells are then delivered into reaction vessels (e.g., wells of a plate) such that each reaction vessel receives, on average, between zero and two of the stem cells or 10,000 to 100,000 of the stem cells, and preferably 10,000-50,000 of the stem cells. The gRNAs may have targeting portions that map to promoter regions of genes associated with a desired phenotype or trait. Each reaction vessel may receive guide RNAs that target either one or a plurality of genes associated with the desired phenotype or trait. For each gene that is targeted, between one and five distinct gRNAs may be provided. Preferably, for each gRNA that is delivered, between about one and about twenty copies of the guide RNA are delivered.

In one approach, genes are targeted individually in high throughput array format (96-well plate, 384-well plate), where activation of a single cell is desired per well. The cells may be delivered into the wells of a plate such that each well receives, on average, between 10,000 to 100,000 cells. In a single gene activation approach, individual sgRNAs per well or two to five pooled sgRNAs per gene per well are used. In another approach, all sgRNAs are pooled and delivered to a whole population of cells. When a viral vector is employed, a multiplicity of infection (MOI) is used, where each cell statistically receives either a single sgRNA or the necessary number of pooled sgRNAs. Targeted gDNA sequencing is used to confirm MOI after transduction.

In an optional workflow, when the starting cells for screening are from an existing iPS cell line, recombinant dCas9-VPR ribonucleoproteins (RNPs) complexed with barcoded sgRNA may be directly delivered to the iPSCs either in pooled or individually arrayed format. Because RNPs are transient (24-72 hours), it is necessary to perform repeat deliveries. However, this approach provides the advantage of temporal control in targeting multiple genes across a time frame (e.g., a few days to a few weeks) to determine the effects of their collective input on producing the desired target phenotype. In the pooled format, the dosage of RNPs may be titered so that each cell statistically receives more or less complex combinations of sgRNAs. In this approach, any iPS cell line can be used as the starting cell type for screening.

### 3. Characterization of Generated Cells

Cell types can be identified by cell traits characteristic of the specific cell type. Furthermore, in addition to the cell traits of a cell type, subtypes have their own cellular traits, as such, cell subtypes can be identified.

Cell traits may include cell morphology, chromosome analysis, DNA analysis, protein expression, RNA expression, enzyme activity, cell-surface markers, or a combination thereof. Characterizing the cells generated to identify their cell type can include analytic methods to assess changes in gene expression and protein expression. Such methods may include one or more of quantifying expression levels via single-cell or bulk RNA-Seq, RT-qPCR, immune staining, immune fluorescence, flow cytometry or evaluating DNA-protein interaction via chromatin immunoprecipitation and DNA sequencing (ChIP-seq).

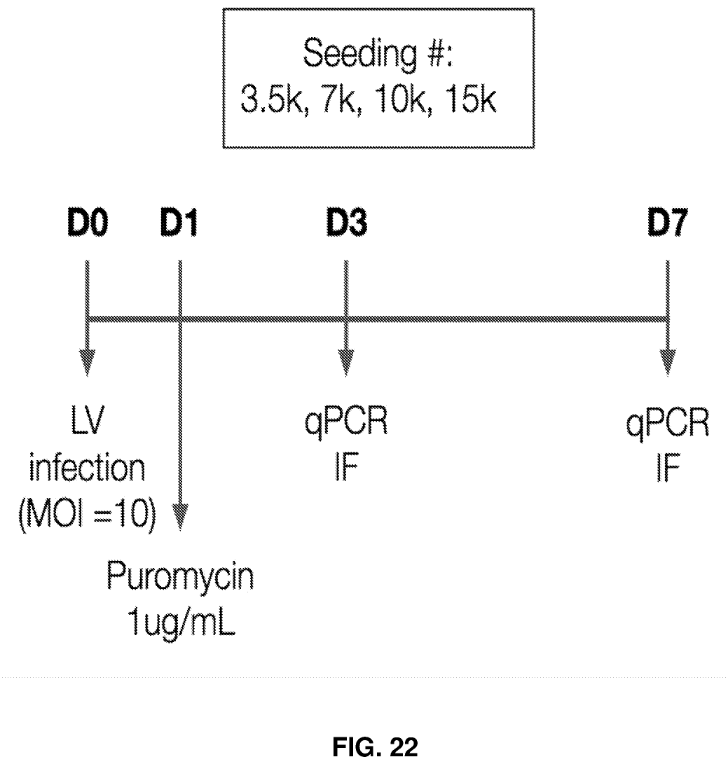

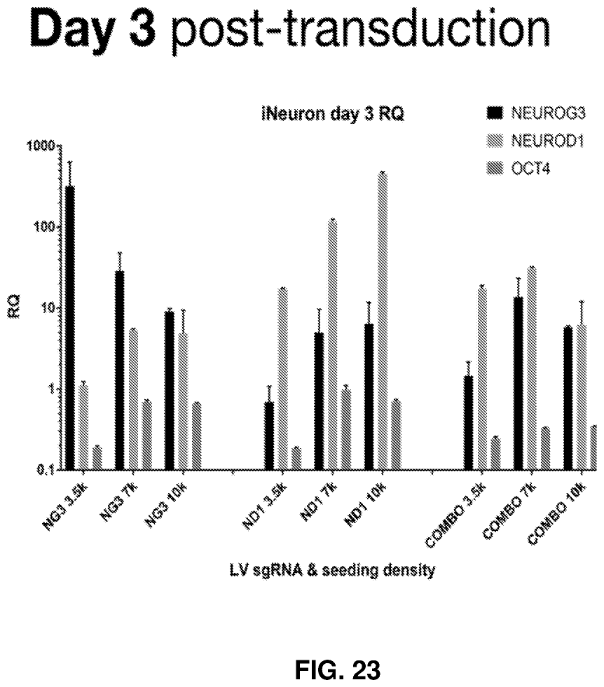

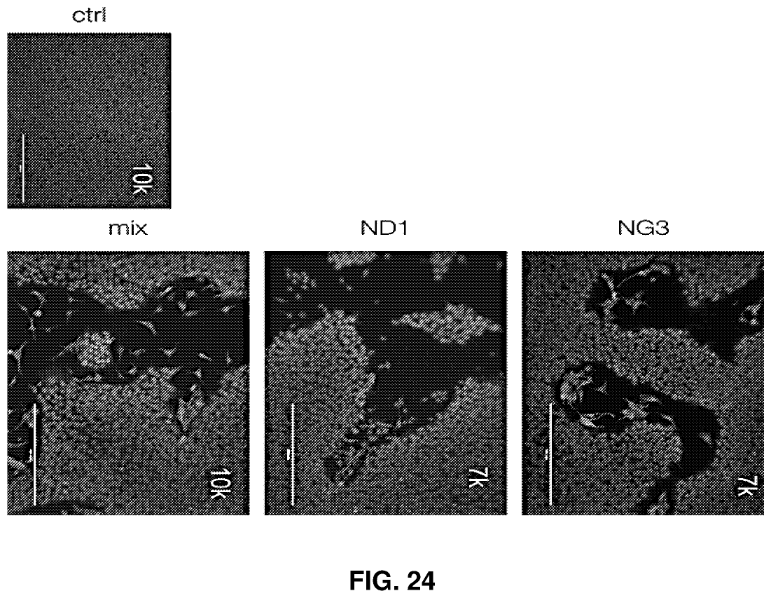

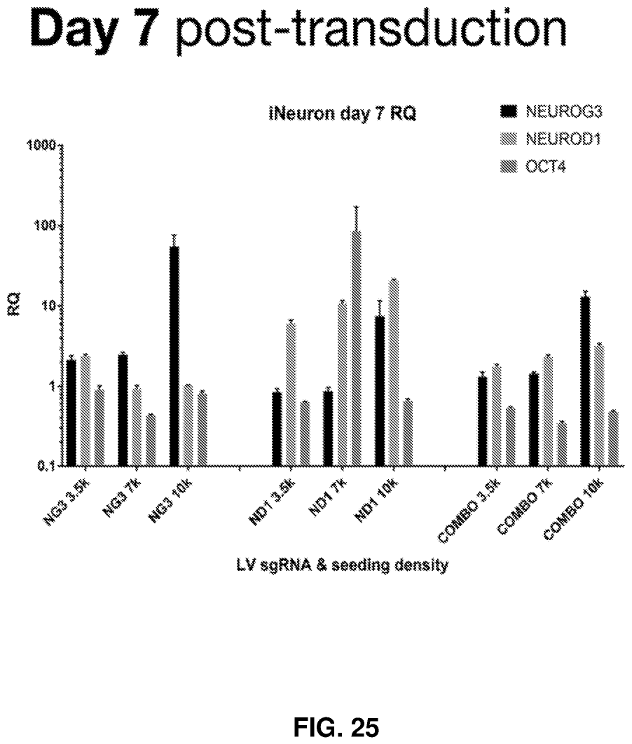

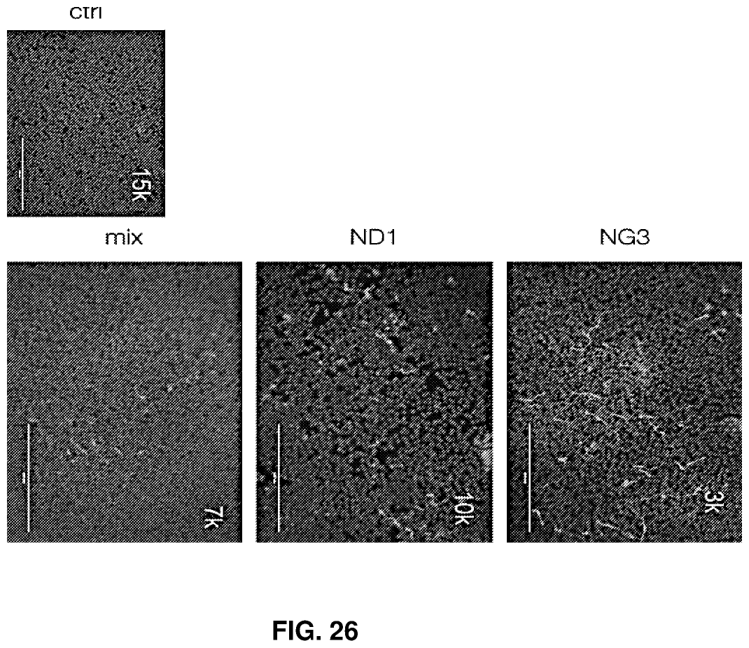

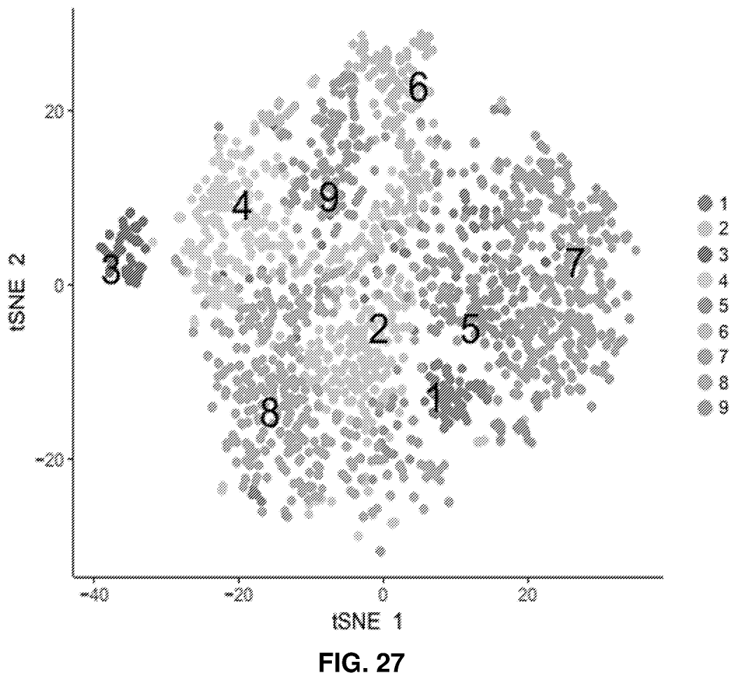

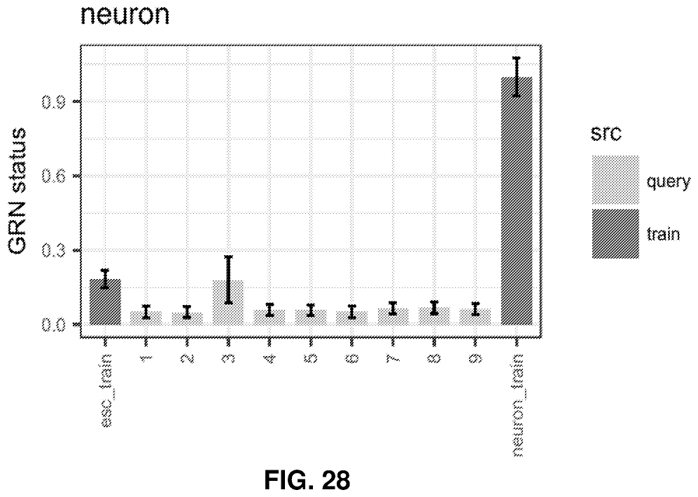

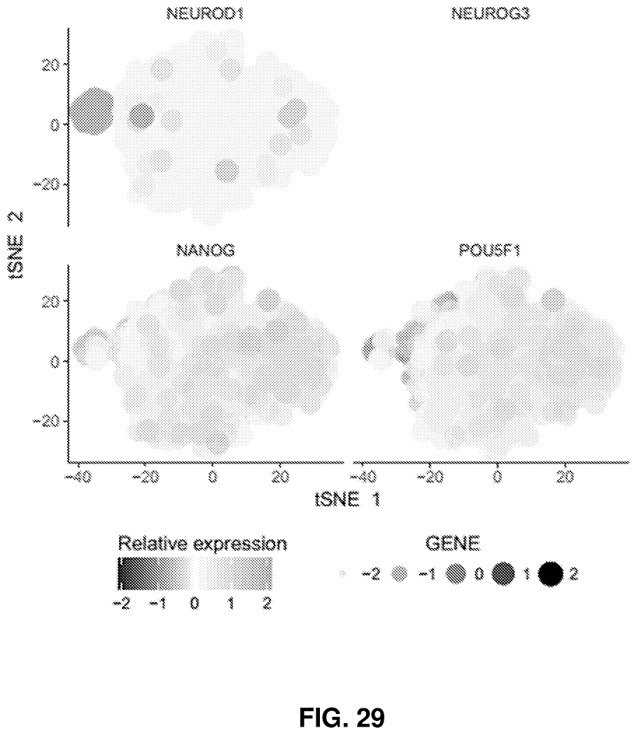

In this example, CRISPRa iPSCs transduced with lentiviral (LV) sgRNAs resulted in the formation of inducible neurons (iNeurons) by day 3. FIG. 22 depicts the changes in gene expression by staining for the neuronal-specific marker, beta III tubulin. Day 3 iNeurons possessed morphological cell traits similar to early-to-committed neuronal precursor cells, along with some mature neurons with extended neurites and arborization. As depicted in FIG. 23 and FIG. 24, the iNeurons morphology compared to mature and varying specialized neurons with longer neurites and extensive arborization at day 7. At day 10, samples were collected for transcriptomic analysis by single-cell RNA-seq (scRNA-seq). As depicted in FIG. 25 and FIG. 26, the day 10 cells were clustered into nine (9) different groups. FIG. 27 depicts the GRN status of the different clusters using methods described herein. Furthermore, FIG. 28 provides the relative expression of the cells, of note is the saturated cluster in NEUROD1. As depicted in FIG. 29, 30% of the cells in Cluster 3 (FIG. 25) are classified as hNbM, which is a subtype of neuroblast, which is consistent with the dopaminergic neuron maturation markers disclosed. Providing that by methods of the invention, the iPSC-derived cells successfully differentiated into neuronal subtypes.

Furthermore, the kinetics of differentiation and ability of iPSCs to specify into a subtype, such as iNeuron subtypes, can be controlled by plating density, whether one or both factors are used (though either alone is sufficient for iNeuron), their relative timing, and duration of differentiation. The order in which factors are selectively turned on and off can also be used to further tune subtype specification and can be identified by methods herein. Embodiments of the disclosure include temporal control of CRISPRa/i+/−TFs. For example, in order to get to the desired synthetic dopaminergic neuron cell, CRISPRa/i may be used against a few targets for the first 2-3 days, followed by CRISPRa/I against some of the same or different genes+/− other genes expressed via PiggyBac TF for another #of days, then CRISPRa/I against some similar or other targets for the remaining #of days of differentiation. Thus, the transcription regulator under guidance of the dCas protein and one or more guide RNAs may result in differentiation of one of the plurality of stem cells when guide RNAs are introduced into at least one of the plurality of stem cells in a temporal sequence. The temporal sequence may include the introduction of a first set of one or more guide RNAs during a first period comprising one or more hours or days followed by introduction a second set of one or more guide RNAs during a second period comprising one or more hours or days. Optionally, the first set of one or more guide RNAs and the second set of one or more guide RNAs comprise wholly different guide RNAs and/or the first period and the second period do or do not overlap in time. Certain embodiments of methods of the disclosure involve using CRISPRa/i against a first set of targets during the first period, the first period comprising at least two days, and using CRISPRa/i against a second set of targets during the second period to differentiate the one of the plurality of stem cells into a dopaminergic neuron cell.

Having previously characterized the effects of NEUROD1 and NEUROG3-mediated rapid differentiation of iPSC with minimal CRISPRa targets and effectors, the identification of additional genes and guide RNAs that could mediate further cell specification to desired phenotypic subtypes was desired. As discussed, identification of the initial gene targets was performed by bioinformatics analysis of a plurality of data, as such additional gene targets and corresponding guide RNA can be identified similarity and repeated until the desired cell type or subtype is identified.

As such, in order to further differentiate the stem cells into the desired subtype of a dopaminergic neuron cell, the cycle of identifying minimal gene targets and sequences of corresponding guide RNAs was repeated.

### 4. Computational Prediction and Identification of Additional Factors

To identify additional genes responsible for differentiation of a subtype the plurality of data (including, for example publically available genomic expression data and scRNA-seq data comprised of midbrain development time courses from mouse, human, and human stem cells (Manno, 2016)) were reanalyzed. For example, a general single-cell trajectory detection algorithm, CellRouter (Lummertz da Rocha, 2018) can be used to reanalyze the data and input the results into the data for further analysis by methods of the invention. However, the outputs from CellRouter are just one data set that can be analyzed be methods of the invention.

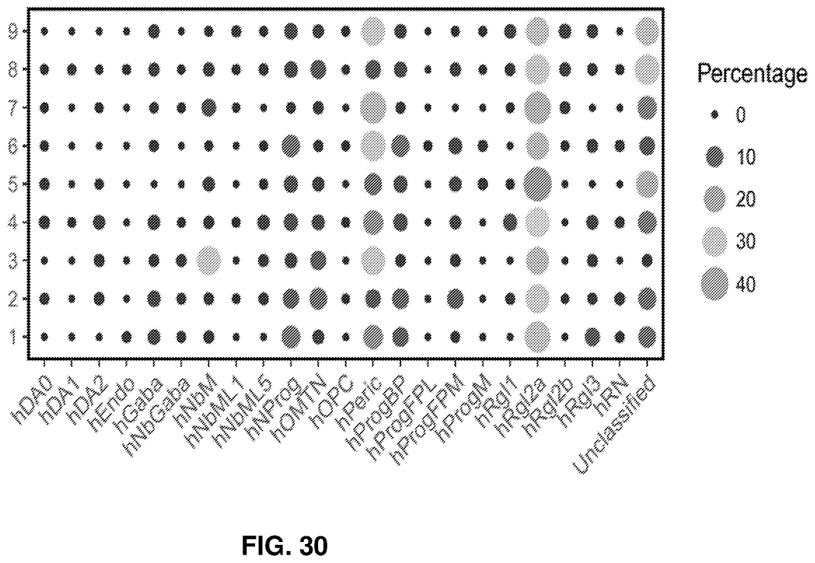

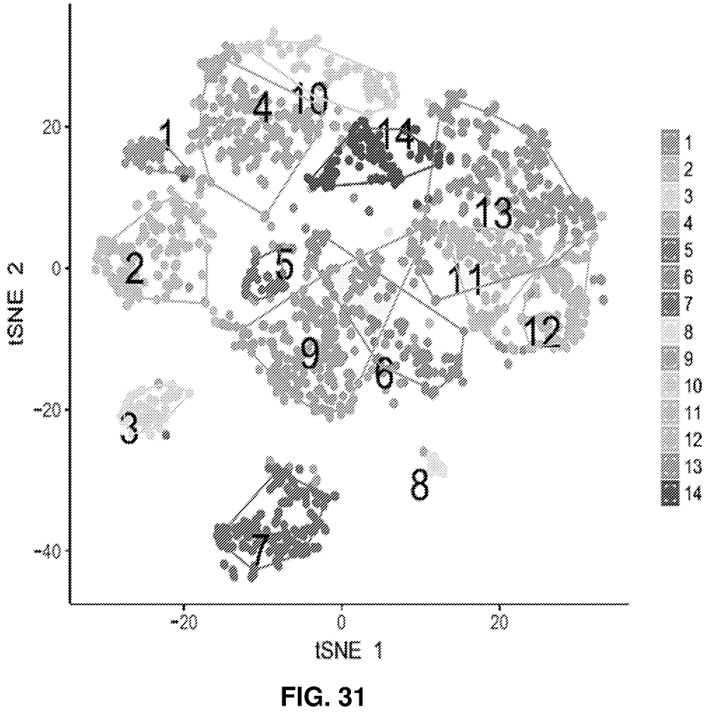

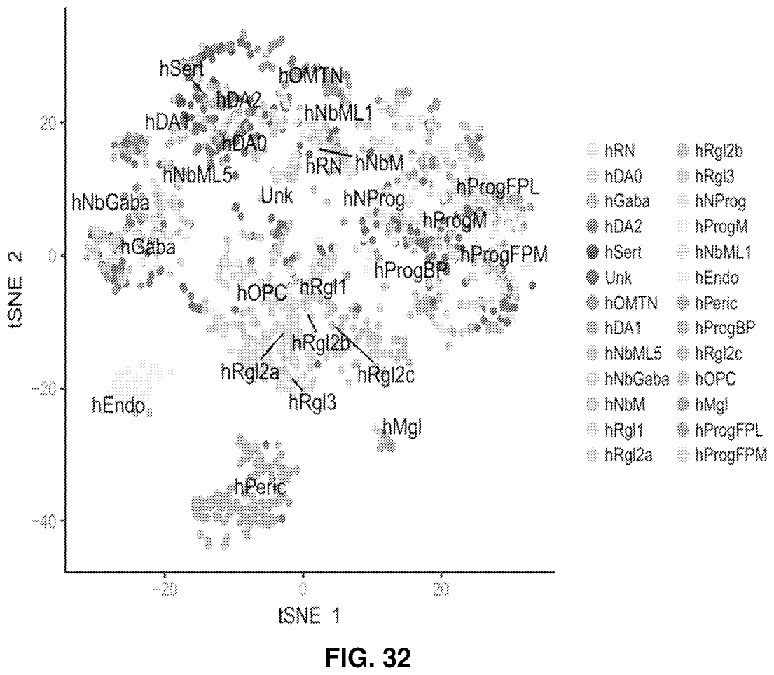

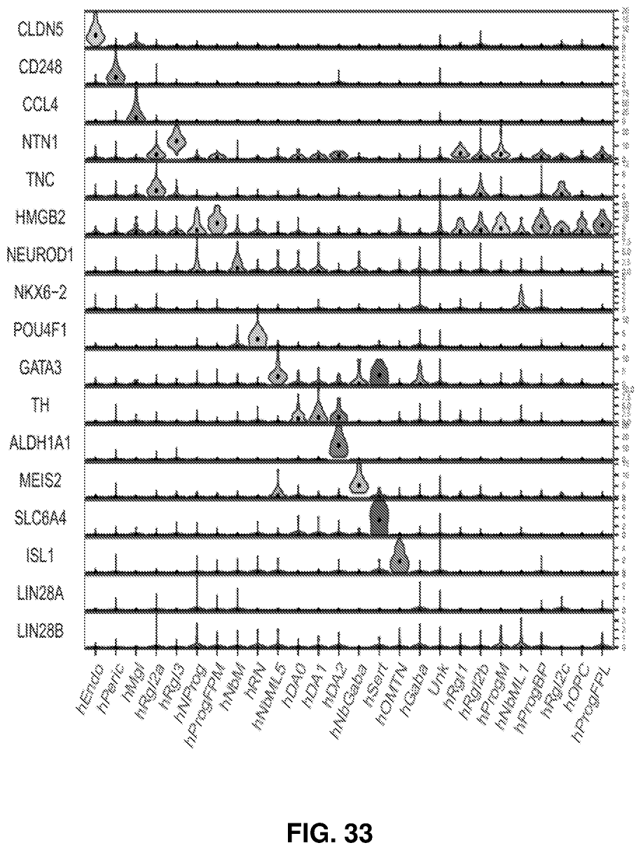

As depicted by FIG. 30, transcriptome analysis was performed on the scRNA-seq data from the developing human midbrain, and the cells were then computationally classified into distinct cell clusters by their transcriptome similarities (cell types 1-12). Furthermore, as depicted in FIG. 31, the cell clusters were then categorized into cell subtypes using previously established gene signatures for 25 cellular sub-identities. FIG. 32 depicts the gene enrichment of the different subtypes, which then allowed for the identification of the gene regulatory networks (GRNs) controlling each specific subtype's function, such as those seen in the immature neural progenitor cells (NPCs) and the more mature dopaminergic (DA) neuron subtypes. The model was then refined by reconstructing each of the identified subtype's differentiation trajectories, as depicted in FIG. 33.

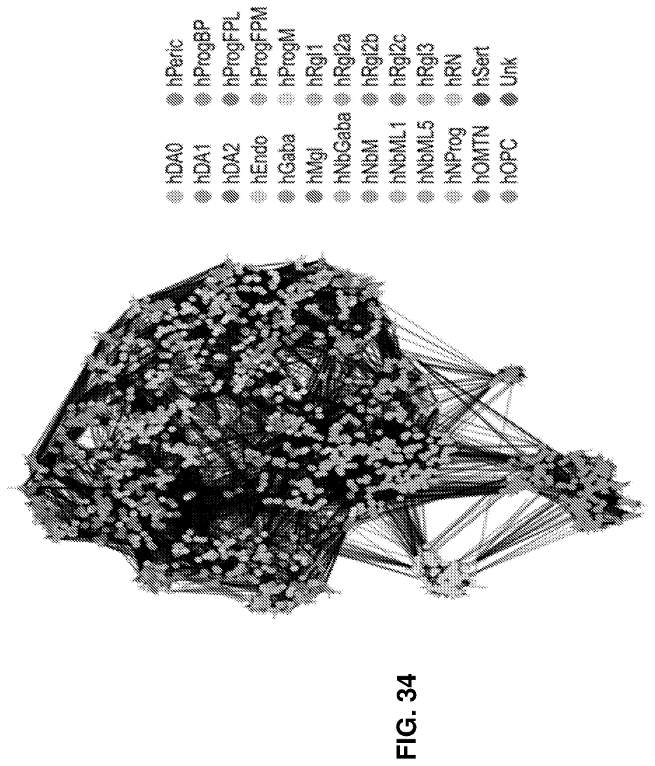

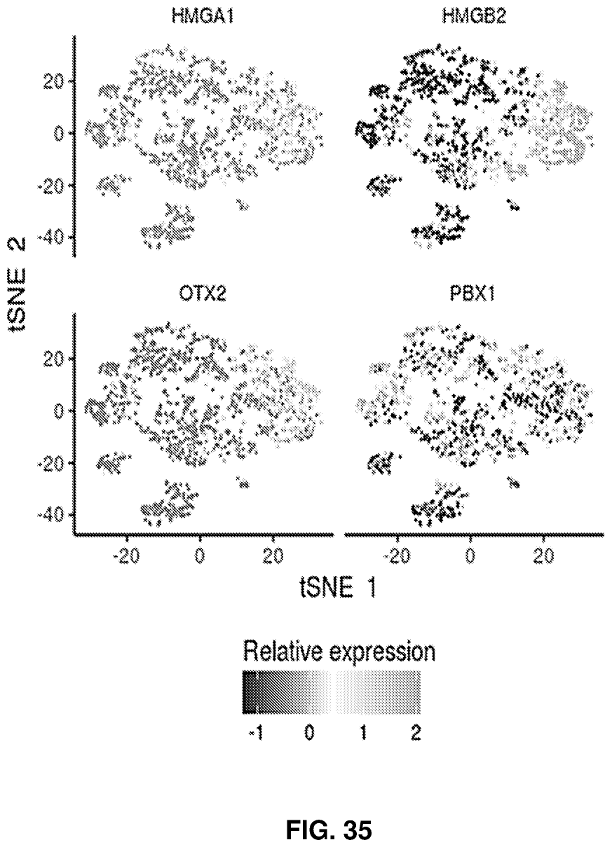

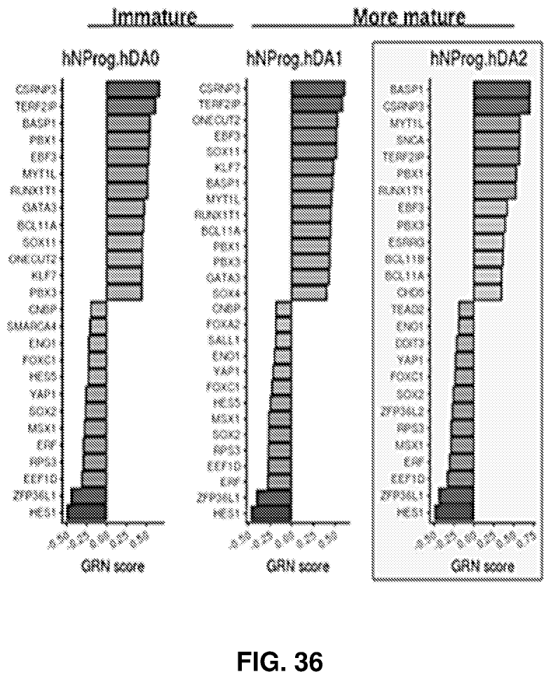

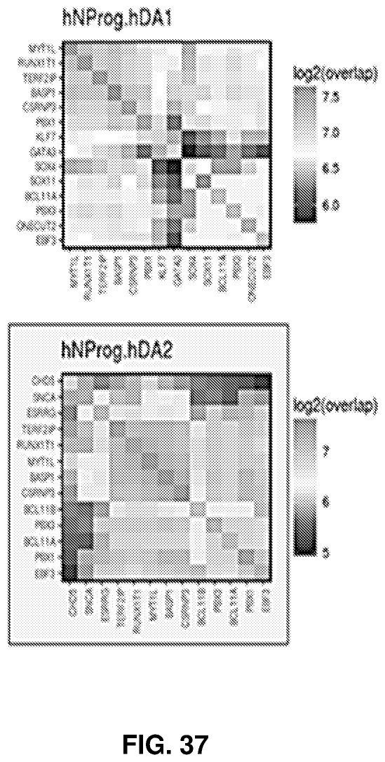

For example, FIG. 34 depicts four (4) t-SNE plots mapping genes (HMGA1, HMGB2, OTX2 and PBX1) previously known to be involved in differentiation from NPCs to dopaminergic neurons and the different subtypes of cells to show the gene expression of those genes in those types of cells. Such a detailed reconstruction of the differentiation pathways from the immature NPCs to both immature (hDA0) and more mature (hDA1 and hDA2) dopaminergic neurons was established by this analysis. The genes associated with the differentiation pathways were then assigned a GRN score using the methods described herein, thereby identifying the top 13 up-regulated and top 13 down-regulated genes responsible for establishing the GRNs responsible for each cellular subtype identity (FIG. 35). These top-level nodes (or genes) were mapped onto downstream networks (FIG. 36), by prioritizing minimal overlap of downstream targets and maximum GRN score, the identification of a minimal set of two genes (BASP1 and SNCA) were predicted to differentiate NPCs into mature dopaminergic neurons (hNProg.hDA2) (FIG. 37).

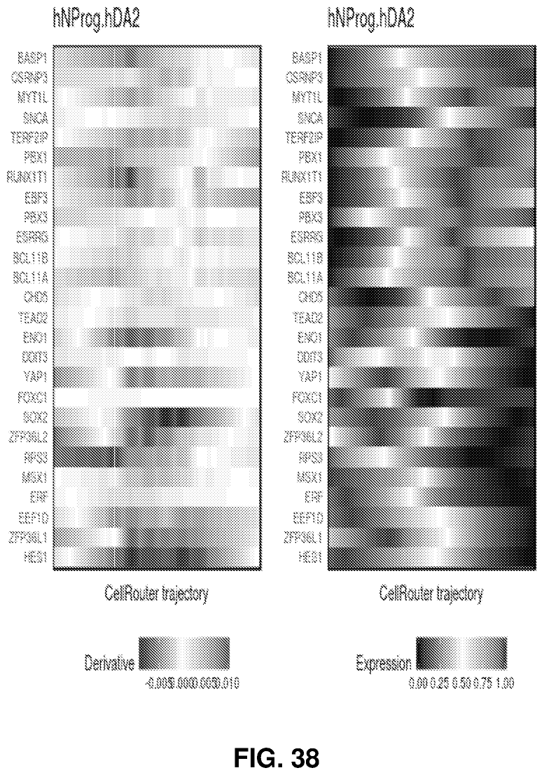

The temporal sequence of gene activation necessary to establish DA neuron identity was then established. Through the trajectory reconstruction analysis, a temporal timing of gene activation based on the identified relationship-based lineage trajectory mapping of the scRNA-seq data set with step-wise progressions through development was established. As shown in FIG. 38, the relative expression levels of the top-level nodes for mature dopaminergic neurons were plotted across time (right), and the derivate of these expression levels (left) identifies inflection points in gene expression. For example, it is identified from these data that to achieve a DA neuron fate from NPCs, over-expression of BASP1, then SNCA is necessary. Based on the system's analysis of the dynamics of GRNs during hDA2 differentiation from NPCs previously identified, MYT1L and BASP3 are predicted to regulate each other. Therefore, overexpression of BASP1 is predicted to induce expression of MYT1L.

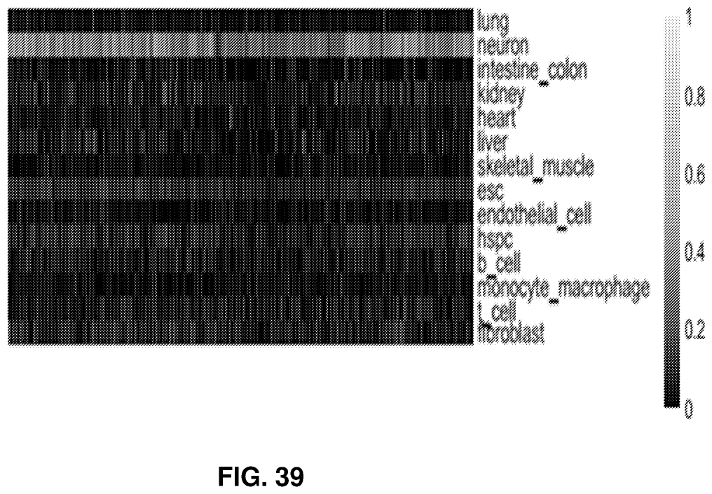

The predicted manipulation of GRNs and resulting cells were computationally verified using the CellNet (Cahan, 2014) system, the results of which are represented in FIG. 39-41. The dataset was processed profiling a time-course (42 e 63 hours) of neuron differentiation of iPSCs to identify whether iPSC-derived neurons are transcriptionally similar to neurons present in the CellNet training dataset. The heat map of FIG. 39 depicts the probability of iPSC-derived neurons relative to neurons in the training dataset (classification scores). For example, values close to 1 indicate that there is a high probability of iPSC-derived neurons to be molecularly similar to neurons in the CellNet training dataset, which represents a broad neuron type.

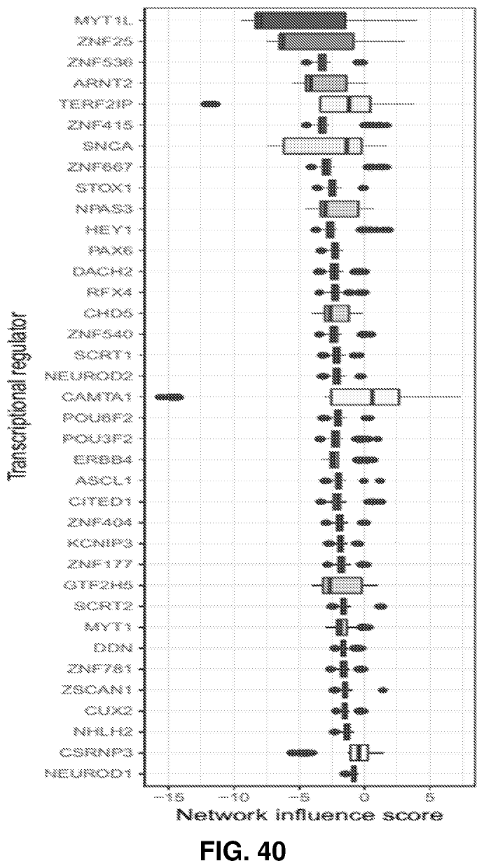

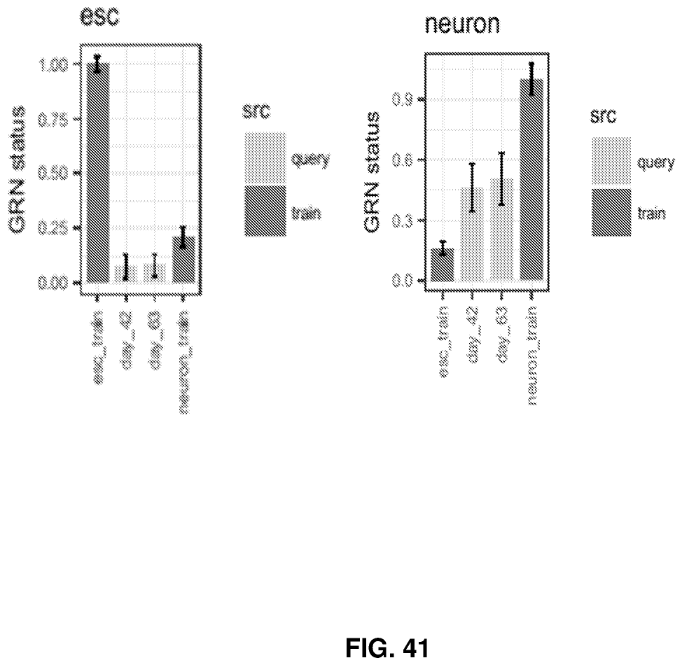

Moreover, reconstruction of the cell-type specific GRNs that represent the ‘identity’ of a particular cell type can be outputted by CellNet. The GRN status quantifies the establishment of a cell-type specific GRN in the query samples, providing enhanced resolution to determine the activation or repression of a source (e.g., iPSC) and target cell type (e.g., neuron) GRN. FIG. 40 depicts the ‘embryonic stem cell’ (ESC) GRN as silenced during iPSC differentiation towards the neuronal fate (left plot), while the neuron GRN is activated, consistent with the induction of the neuronal fate. Finally, the Network Influence Score (NIS), presented in FIG. 41, aim at identifying transcriptional regulators to enhance cell engineering by quantifying the extent of dysregulation of these transcriptional regulators relative to the training dataset. This analysis provides that iPSC-derived neurons lack expression of critical neuron genes such as MYT1L and SNCA. This analysis verified that the predicted genes and temporal sequence of expression would generally produce neurons. Unfortunately, the CellNet system is unable to map to individual neural sub-identities.

However, the presently disclosed methods and systems, are able to receive this data (e.g., GRNs, genes, temporal expression of the genes as outputted by the CellRouter system) and continuously train the data set to classify cellular subtypes and verify resulting cell types, subtypes and phenotypes computationally, which is a vast improvement over the prior art. As described throughout, the system receives data from many other data sources and internally generated scRNA-seq data. Upon verification of the subtype in silico, the sequences of a minimum number of guide RNAs for each of the identified additional gene targets are identified using the methods described herein system described herein.

### 5. Iteration Until the Desired Cellular Phenotype is Achieved

Utilizing the verified additional gene predictions from the system and their corresponding guide RNAs, dCas9-VPR iPSCs were transduced using the methods described herein with lentiviral sgRNAs activating NEUROD1, BASP1, and SNCA in different permutations and temporal combinations as identified by methods of the invention. The result of which was the specification of dopaminergic neurons from stem cells.

If the desired phenotype is not achieved after a single process through the disclosed system and methods, then the “design-build-test” cycles (i.e., steps 2-4 of this example) are repeated until the desired cellular type is identified using the disclosed systems and methods.

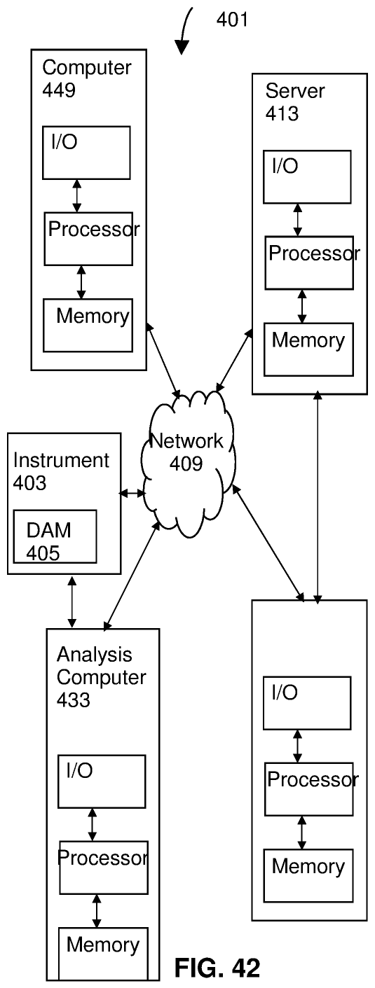

As contemplated throughout, one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus. FIG. 42 diagrams the system 401 for identifying the minimum number of targets to specify cell fate. The system 401 includes at least one computer 449, such as a laptop or desktop computer, than can be accessed by a user to initiate methods of the invention and obtain results. The system 401 preferably also includes at least one server sub-system 413 and either or both of the computer 449 and the server sub-system 413 may include and provide the machine learning system of the invention. The server subsystem 413 may have a dedicated terminal computer 467 for accessing the server sub-system 413. Additionally, in some embodiments, the system 401 operates in communication with a laboratory, which may include an analysis instrument 403 such as a gene expression instrument. The analysis instrument 403 may have its own data acquisition module 405, such as, for example, the electronic instruments of a single-cell RNA sequencer, an RNA multiplex sequencer (e.g., nCounter), a microarray, or RT-qPCR. The instrument 403 may have its own built-in or connected instrument computer 433. Any or all of the computer 449, server subsystem 413, terminal computer 467, instrument 403, and instrument computer 433 may exchange data over communications network 409, which may include elements of a local area network (LAN), a wide area network (WAN) the Internet, or combinations thereof. Each of computer 449, server subsystem 413, terminal computer 467, and instrument computer 433, when included, preferably includes at least one processor coupled to one or more input/output devices and a tangible, non-transitory memory subsystem. The I/O devices may include one or more of: monitor, keyboard, mouse, trackpad, touchpad, touchscreen, Wi-Fi card, cellular antenna, network interface cards, or others. The memory subsystem preferably includes one or more of RAM and a disc drive, such as a magnetic hard drive or solid state drive.

Memory according to the invention can include a machine-readable medium on which is stored one or more sets of instructions (e.g., software) embodying any one or more of the methodologies, functions or outputs of the methodologies described herein. The software may also reside, completely or at least partially, within the main memory and/or within the processor during execution thereof by the computer system, the main memory and the processor also constituting machine-readable media. The outputs include programs for the temporal expression of the identified gene targets to achieve cell fate specification. Optionally, these programs also include guide RNA sequences respective to the gene targets. The software may further be transmitted or received over a network via the network interface device. Ultimately, the systems disclosed herein, encompasses a generalizable iterative system capable of identifying minimal target genes and their minimal effectors (i.e., guide RNAs) capable of directing cell differentiation between any given cell identities (e.g. iPSCs, NPCs, and DA neurons), ultimately directing cell fate of a cell through machine learning and experimental validation.

Furthermore, sets of genes, or gene modules, can be identified as being responsible for affecting a certain phenotype of cell. The gene modules identified can be utilized in any cell type to effectuate the desired phenotype. In contrast to the prior art, the methods disclosed enable the generalizable selection of a minimal number of targets with a corresponding minimum number of gRNAs that direct cell fate specification with additional maturation through identification of additional targets via machine learning methods in iterative design-build-test cycles.

Starting Cell Types

Methods of the invention may be applied to, but are not limited to the following example cell types: Human BC-1 Cells, Human BJAB Cells, Human IM-9 Cells, Human Jiyoye Cells, Human K-562 Cells, Human LCL Cells, Mouse MPC-11 Cells, Human Raji Cells, Human Ramos Cells, Mouse Ramos Cells, Human RPMI8226 Cells, Human RS4-11 Cells, Human SKW6.4 Cells, Human, Dendritic Cells, Mouse P815 Cells, Mouse RBL-2H3 Cells, Human HL-60 Cells, Human NAMALWA Cells, Human Macrophage Cells, Mouse RAW 264.7 Cells, Human KG-1 Cells, Mouse M1 Cells, Human PBMC Cells, Mouse BW5147 (T200-A)5.2 Cells, Human CCRF-CEM Cells, Mouse EL4 Cells, Human Jurkat Cells, Human SCID.adh Cells, Human U-937 Cells, Human HOS Cells Human Saos-2 Cells, Human U-2 OS Cells, Human MH7A Cells, Mouse 3T3-L1 Cells, Human BJ Cells Monkey COS-7 Cells, Human Neonatal Dermal Fibroblast Cells, Horse Embryonic Dermal Fibroblast Cells (NBL-6), Mouse Embryonic Fibroblast Cells (MEF), Human HT-1080 Cells, Human, IMR-90 Cells, Mouse L-929 Cells, Mouse NIH-3T3 Cells, Mouse PA317 Cells, Monkey, Vero Cells, Human WI-38 Cells, Mouse b-END.3 Cells, Human Endothelial Cells, Human HUVEC Cells, Rat PC-12 Cells, Human 253J Cells, Human J82 Cells, Human RT4 Cells, Human T24 Cells, Mouse F9 Cells, Mouse P19 Cells, Human ARPE-19 Cells, Human COLO 201 Cells, Human HCT 116 Cells, Human HCT15 Cells, Human HT-29 Cells, Human RKO Cells, Human SW480 Cells, Human WiDr Cells, Human 293A Cells, Hamster BHK-21 Cells, Human HEK 293 Cells, Canine, MDCK Cells, Rat NRK Cells, Human ChangX-31 Cells, Rat H-4-II-E Cells, Human Hep G2 Cells, Human Hep3B Cells, Human SK-HEP-1 Cells, Human SNU-387 Cells, Human BT-20 Cells, Human, HCC1937 Cells, Human Hs-578T Cells, Human Mammary Epithelial Cells, Human MCF7 Cells, Human MCF-ADR Cells, Human MDA-MB-231 Cells, Human SK-BR-3 Cells, Human T-47D Cells, Chinese Hamster CHO DG44 Cells, Chinese Hamster CHO-K1 Cells, Human SK-OV-3 Cells, Human, BxPC-3 Cells, Human PANC-1 Cells, Rat GH3 Cells, Human DU 145 Cells, Human LNCaP Cells, Human TSU-Pr1 Cells, Human PC-3 Cells, Human A549 Cells, Human BEAS-2B Cells, Human NCI-H23 Cells, Human NCI-H69 Cells, Human Calu-3 Cells, Human G-361 Cells, Human HN3 Cells, Human MEWO Cells, Human ARO Cells, Human FRO Cells, Human NPA Cells, Human A-431 Cells, Human HeLa Cells (ATCC), Human C-33 A Cells, Rat Cardiomyocyte Cells, Mouse, C2C12 Cells, Rat L6 Cells, Human Aortic Smooth Muscle Cells, Rat Astrocyte Cells, Rat Cortical, Astrocyte Cells, Rat C6 Glial Cells, Mouse Glial Cells, Rat Glial Precursor Cells, Human T98G Cells, Human U-87 MG Cells, Human SH-SY5Y Cells, Human SK-N-MC Cells, Rat Primary Cortical, Neuron Cells, Mouse GT1-1 Cells, Mouse GT1-7 Cells, Rat HiB5 Cells, Rat Primary Hippocampal Neuron Cells, Rat SCN2.2 Cells, Rat F-11 Cells, Human SW-13 Cells, Human SV40 MES 13 Cells, Human Mesenchymal Stem Cells (hMSC), Human BGO1V Embryonic stem Cells, Human H9, Embryonic stem Cells, Mouse Embryonic stem Cells, Human adipose-derived stem cells (ADSC), Human Neural Stem Cells, Rat Neural Stem Cells, Fibroblast (iPSC)—Fibroblast, CD34+, Adipose, Derived Stem Cells, Adrenal Cortical Cells, Alpha Cells, Annulus Fibrosus Cells, Astrocytes, Beta Cells, Chondrocytes, Endothelial Cells, Epithelial Cells, Fibroblasts, Hair Cells, Hematopoietic Stem Cells, Immune Cells, Keratinocytes, Keratocytes, Melanocytes, Meningeal Cells, Mesangial Cells, Mesenchymal Stem Cells, Muscle Myoblasts, Muscle Sattellite Cells, Nucleus Pulposus Cells, Osteoblasts, Pericytes, Perineurial Cells, Schwann Cells, Skeletal Muscle Cells, Smooth Muscle Cells, Stellate Cells, Synoviocytes, Thymic Epithelial Cells, Trabecular and Meshwork Cells, Trophoblasts, Bone Marrow CD34+ Stem/Progenitor Cells, Bone Marrow Mononuclear Cells, Cord Blood Mononuclear Cells, Cord Blood CD34+ Stem/Progenitor Cells, Cord Blood CD34-Depleted MNC, Cord Blood CD3+ Pan T Cells, Cord Blood CD4+ Helper T Cells, Cord Blood CD8+ Cytotoxic T Cells, Cord Blood CD4+/CD45RA+ Naive T Cells, Cord Blood CD14+ Monocytes, Cord Blood CD56+ Natural Killer Cells, Cord Blood Plasma, Diseased Peripheral Blood CD19+/CD5+ B Cells, Diseased Bone Marrow CD19+/CD5+ B Cells, Diseased Bone Marrow MNC, Diseased Peripheral Blood MNC, Diseased Bone Marrow MNC, Diseased Peripheral Blood MNC, Diseased Peripheral Blood MNC, Diseased Peripheral Blood MNC, Diseased Peripheral Blood MNC, Diseased Peripheral Blood MNC, Diseased Bone Marrow MNC, Diseased Peripheral Blood MNC, Diseased Peripheral Blood Plasma, Mobilized Peripheral Blood Mononuclear Cells, Mobilized Peripheral Blood CD34+ Stem/Progenitor Cells, Mobilized Peripheral Blood CD14+ Monocytes, Bone Marrow Mesenchymal Stem/Stromal Cells, Diseased Peripheral Blood MNC, Peripheral Blood Monocyte-Derived Dendritic Cells, Peripheral Blood Monocyte-Derived Macrophages, Peripheral Blood Mononuclear Cells (PBMC), Peripheral Blood CD3+ Pan T Cells, Peripheral Blood CD4+ Helper T Cells, Peripheral Blood CD8+ Cytotoxic T Cells, Peripheral Blood CD4+/CD25+ Regulatory T Cells, Peripheral Blood CD4+/CD45RA+/CD25− Naive T Cells, Peripheral Blood CD8+/CD45RA+ Naive Cytotoxic T Cells, Peripheral Blood CD4+/CD45RO+ Memory T Cells, Peripheral Blood CD19+ B Cells, Peripheral Blood CD19+/IgD+ Naive B Cells, Peripheral Blood CD14+ Monocytes, Peripheral Blood CD56+ Natural Killer Cells, Peripheral Blood Basophils, Peripheral Blood Eosinophils, Peripheral Blood Neutrophils, Peripheral Blood Plasma, Peripheral Blood Platelets, Peripheral Blood Mature Erythrocytes (RBC), Peripheral Blood CD34+ Stem/Progenitor Cells, Diseased Peripheral Blood MNC, Diseased Peripheral Blood MNC, Diseased Peripheral Blood MNC, Diseased Peripheral Blood MNC, Induced pluripotent cell (iPS cells), “True” embryonic stem cell (ES cells) derived from embryos, Embryonic stem cells made by somatic cell nuclear transfer (ntES cells), Embryonic stem cells from unfertilized eggs (parthenogenesis embryonic stem cells, or pES cells) Totipotent, Zygote, Spore, Morula,

Pluripotent, Embryonic stem cell, Callus, Multipotent cells, Progenitor cells, Endothelial stem cells, Hematopoietic stem cells, Mesenchymal stem cells, Neural stem cell, Neural Progenitor cells, Unipotent Precursor cells, Oligodendrocyte precursor cell, Myeloblast, Thymocyte, Meiocyte, Megakaryoblast, Promegakaryocyte, Melanoblast, Lymphoblast, Bone marrow, precursor cells, Normoblast, Angioblast (endothelial precursor cells), Myeloid precursor cells, Neural Stem Cells, Neural Porgenitor Cells, Neural Precursor Cells, Discovery, Intestinal enteroendocrine cells, K cell, L cell, I cell, G cell, Enterochromaffin cell, N cell, S cell, D cell, M cell, Gastric enteroendocrine cells, Pancreatic enteroendocrine cells, Alpha cells, Beta Cells, Delta Cells, PP cells, Epsilon Cells, Hepatocytes, Kupffer Cells, Stellate (Ito) Cells, Liver Sinusoidal Endothelial Cells, Neurons (unipolar, bipolar, multipolar, Golgi I and II, Anaxonic, peuodounipolar), Basket Cells, Betz Cells, Lugaro Cells, Medium spiny neurons, Purkinje Cells, Pyramidal cells, Renshaw cells, Unipolar brush cells, Granule Cells, Anterior Horn Cells, Spindle Cells, Salivary gland mucous cell (polysaccharide-rich secretion), Salivary gland number 1 (glycoprotein enzyme-rich secretion), Von Ebner's gland cell in tongue (washes taste buds), Mammary gland cell (milk secretion), Lacrimal gland cell (tear secretion), Ceruminous gland cell in ear (earwax secretion), Eccrine sweat glandering dark cell (glycoprotein secretion), Eccrine sweat gland clear cell (small molecule secretion), Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Insolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion, Paneth cell of small intestine (lysozyme secretion), Type II pneumocyte of lung (surfactant secretion), Club cell of lung, Anterior pituitary cells, Somatotropes, Lactotropes, Thyrotropes, Gonadotropes, Corticotropes, Intermediate pituitary cell, secreting melanocyte-stimulating hormone, Magnocellular neurosecretory cells, nonsecreting oxytocin, secreting vasopressin, Gut and respiratory tract cells, secreting serotonin, secreting endorphin, secreting, somatostatin, secreting gastrin, secreting secretin, nonsecreting cholecystokinin, secreting insulin, secreting glucagon, nonsecreting bombesin, Thyroid gland cells, Thyroid epithelial cell, Parafollicular cell, Parathyroid gland cells, Parathyroid chief cell, Oxyphil cell, Adrenal gland cells, Chromaffin cells, secreting steroid hormones (mineralocorticoids and gluco corticoids), Leydig cell of testes secreting testosterone, Theca interna cell of ovarian follicle secreting estrogen, Corpus luteum cell of ruptured ovarian follicle secreting progesterone, Granulosa lutein cells, Theca lutein cells, Juxtaglomerular cell (renin secretion), Macula densa cell of kidney, Peripolar cell of kidney, Mesangial cell of kidney, Pancreatic islets (islets of Langerhans), Alpha cells (secreting glucagon), Beta cells (secreting insulin and amylin), Delta cells (secreting somatostatin), PP cells (gamma cells) (secreting pancreatic polypeptide), Epsilon cells (secreting ghrelin), Erythrocyte (red blood cell), Megakaryocyte (platelet precursor), Monocyte (white blood cell), Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Hybridoma cell, Mast cell, Helper T cell, Suppressor T cell, Cytotoxic T cell, Natural killer T cell, B cell, Natural killer cell, Reticulocyte, Hematopoietic stem cells and committed progenitors for the blood and immune system (various types).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A system for image-driven cell manufacturing comprising:
a substrate having a surface on which cells are disposed;
an imager configured to image cells on the substrate at high resolution; and
a pulsed laser scanner that directs laser pulses to the substrate under the cells.

2. The system of claim 1, further comprising:
a processor; and
a computer-readable storage device containing instructions that when executed by the processor cause the system to:
receive data from plurality of sources;
perform an analysis on the data to identify targets related to cell differentiation of a desired cell type; and
direct laser pulses from the pulsed laser scanner to the substrate under the targets.

3. The system of claim 1, wherein the imager and the pulsed laser scanner are communicatively coupled to the processor.

4. The system of claim 1, wherein the substrate comprises an absorbing layer that absorbs laser pulses, the absorbing layer disposed between the surface on which cells are disposed and the pulsed laser scanner.

5. The system of claim 4, wherein the absorbing layer is a partially absorbing layer.

6. The system of claim 4, wherein the absorbing layer comprises titanium, gold, or a combination thereof.

7. The system of claim 4, wherein the absorbing layer absorbs transmission at one or more imaging wavelengths in a range of about 400 nm to about 700 nm.

8. The system of claim 1, wherein the substrate does not leach or ablate into cell culture.

9. The system of claim 1, wherein the substrate comprises an antireflection layer on a surface of the substrate opposite the surface on which cells are disposed.

10. The system of claim 1, wherein the pulsed laser scanner transmits laser pulses at a wavelength of greater than about 500 nm.

11. The system of claim 1, wherein the pulsed laser scanner has a pulse frequency of greater than about 10 kHz.

12. The system of claim 2, wherein the targets are mammalian genes.

13. The system of claim 12, wherein the mammalian genes correspond to a species selected from mouse, human, and a combination thereof.

14. A method for image-driven cell manufacturing comprising:
disposing cells on a substrate;
imaging cells on the substrate at high resolution to create images;
computing cell characteristics from the images to identify selected cells; and
directing a pulsed laser scanner to deliver laser pulses to the substrate under the selected cells, thereby manufacturing cells.

15. The method of claim 14, wherein a coating layer of the substrate partially absorbs laser pulses to convert optical energy into microbubble formation.

16. The method of claim 14, further comprising destroying selected cells with the microbubble formation.

17. The method of claim 14, further comprising removing selected cells with the microbubble formation.

18. The method of claim 14, further comprising porating selected cells with the microbubble formation.

19. The method of claim 18, wherein porating selected cells is a temporary poration.

20. The method of claim 19, further comprising introducing biological cargo to the selected cells during the temporary poration.

21. The method of claim 20, further comprising introducing fluorescent indicator molecules with the biological cargo.

22. The method of claim 21, further comprising imaging the selected cells for presence of the fluorescent indicator molecules, thereby verifying delivery of the biological cargo to the selected cells.

23. The method of claim 19, further comprising delivering barcodes to the cells during the temporary poration, wherein the barcodes are based on cell characteristics from the cell images.

24. The method of claim 23, further comprising distinguishing barcoded cells in downstream analysis.

25. The method of claim 14, wherein computing cell characteristics comprises performing an analysis on data received from a plurality of sources to identify selected cells related to cell differentiation of a desired cell type.

26. The method of claim 14, wherein computing cell characteristics comprises:
determining a minimum number of genes required for differentiation of a stem cell into a selected cell type;
exposing said stem cell to a Cas endonuclease and associated guide RNAs directed at a portion of said genes; and
identifying members of said selected cell type.

27. The method of claim 26, wherein laser pulses are delivered to isolate the members.

28. The method of claim 26, wherein the members are identified by comparing cell traits of the members to the specific cell traits of the cell type.
